(19) United States
(12) Patent Application Publication
(10) Pub. No.: US 2003/0162254 A1
(43) Pub. Date: Aug. 28, 2003

(54) NaPi type IIb polypeptides and methods for making and using same

(76) Inventor: Brian E. Peerce, Friendswood, TX (US)

Correspondence Address: Braman & Rogalskyj, LLP, P.O. Box 352, Canandaigua, NY, 14424-0352, US

(21) Appl. No.: 10/345,071

(22) Filed: Jan. 15, 2003

Related U.S. Application Data
(60) Provisional application No. 60/349,280, filed on Jan. 15, 2002.

(51) Int. Cl. C12P 21/02; C12N 5/06; C07K 14/47; C07H 21/04

(52) U.S. Cl. 435/69.1; 435/320.1; 435/325; 530/350; 536/23.5

(57) ABSTRACT

Disclosed are isolated NaPiIIb polypeptides. Also disclosed are methods for screening a test compound for its ability to bind to or otherwise affect the function of Na⁺/phosphate cotransporter. The method includes providing an isolated NaPiIIb polypeptide; contacting the isolated NaPiIIb polypeptide with the test compound; and determining whether the test compound binds to or otherwise affects the function of isolated NaPiIIb polypeptide. Antibodies and fragments thereof specific for a isolated NaPiIIb polypeptide are also disclosed.

This application claims the benefit of U.S. Provisional Patent Application Serial No. 60/349,280, filed Jan. 15, 2002, which is hereby incorporated by reference.

### FIELD OF THE INVENTION

The present invention is directed to isolated NaPiIIb polypeptides and to methods for making and using same.

### BACKGROUND OF THE INVENTION

Throughout this application various publications are referenced. The disclosures of each of these publications, in their entireties, are hereby incorporated by reference in this application.

In the mammalian small intestine active uptake of dietary phosphorus is coupled to Na⁺ uptake by the brush border membrane Na⁺/phosphate cotransporter. The Na⁺/phosphate cotransporter utilizes the Na⁺ gradient across the enterocyte membrane to couple uphill transport of phosphate across the luminal membrane. Our understanding of the mechanisms involved in ion binding, ion coupling, and ion transport and release are limited by the absence of structural data. Na⁺ is thought to be the obligate or preferred first substrate (Bernier et al., Biochem. J., 160:467-474 (1976); Beliveau et al., J. Biol. Chem, 262:16885-16891 (1987); Peerce, Am. J. Physiol., 256:G645-G652 (1989); and Peerce, J. Membr. Biol., 110:189-197 (1989)). Na⁺ binding to the cotransporter induces a conformational change resulting in high affinity phosphate binding to the cotransporter (Peerce, Am. J. Physiol., 256:G645-G652 (1989); Peerce, J. Membr. Biol., 110:189-197 (1989); Amstutz et al., Am. J. Physiol., 248:F705-F710 (1985); Hoffmann et al., Pflugers Arch., 362:147-156 (1976); Cheng et al., J. Biol. Chem., 256:1556-1564 (1981); and Mohrmann et al., Am. J. Physiol., 250:G323-G330 (1986)). Following Na⁺ binding, phosphate addition results in a second conformational change (Peerce, Biochim. Biophys. Acta, 1323:45-56 (1997)). The role of the (Na⁺+phosphate)-induced conformational change has been suggested to involve vectorial ion transport and the release of Na⁺ (Peerce, Biochim. Biophys. Acta, 1323:45-56 (1997); and Biber et al., Kid. Int., 49:981-985 (1997)).

The intestinal brush border membrane Na⁺/phosphate cotransporter is a member of the NaPi type II cotransporter family. NaPi type II cotransporter family includes the renal PTH-sensitive Na⁺/phosphate cotransporter, NaPiIIa, and the intestinal apical membrane Na⁺/phosphate cotransporter, NaPiIIb. The intestinal brush border membrane Na⁺/phosphate cotransporter, NaPiIIb shares 57% homology with the renal cotransporter, NaPiIIa (Hilfiker et al., Proc. Natl. Acad. Sci. (USA), 95:14564-14569 (1998)). The NaPi family of Na⁺/phosphate cotransporters has been the subject of recent reviews (Murer et al., Physiol. Rev., 80:1373-1409 (2000); and Werner et al., J. Exp. Biol., 201:3135-3142 (1998)).

The protein domains and amino acid residues involved in ion binding and transport are not known. Previous approaches to address these questions have utilized chemical modification reagents and site-directed mutagenesis. Chemical modification studies have identified classes of amino acids involved in Na⁺-dependent phosphate uptake in intestinal and renal brush border membrane vesicles (Peerce, Am. J. Physiol., 256:G645-G652 (1989); and Peerce, J. Membr. Biol., 110:189-197 (1989); Wuarin et al., Biochim. Biophys. Acta, 981:185-192 (1989); Peerce et al., Miner. Electrolyte Metab., 16:125-129 (1990); and Strevey et al., Biochim. Biophys. Acta, 1106:110-116 (1992)). Substrate-induced protection against chemical reagent modification and inhibition of Na⁺-dependent phosphate uptake has suggested that tyrosines (Peerce, J. Membr. Biol., 110:189-197 (1989); and Wuarin et al., Biochim. Biophys. Acta, 981:185-192 (1989)) and arginines (Peerce, Am. J. Physiol., 256:G645-G652 (1989); Peerce et al., Miner. Electrolyte Metab., 16:125-129 (1990); and Strevey et al., Biochim. Biophys. Acta, 1106:110-116 (1992)) are located near the cotransporter substrate sites. These studies did not identify the amino acid residues labeled or determine the role of these residues in cotransporter function.

Site-directed mutagenesis studies of rat NaPiIIa have identified 2 tyrosines which appear to be involved in membrane insertion/retrieval and Na⁺-dependent phosphate uptake (Hernando et al., J. Membr. Biol., 168:275-282 (1999)). Y₄₀₂ may be involved in membrane insertion and retrieval. Y₅₀₉ may be involved in cotransporter function. Y₅₀₉ (Y₅₂₅ mouse NaPiIIb) and Y₄₀₂ (Y₄₁₇ mouse NaPiIIb) are conserved in NaPiIIb. Rat R₅₁₀ (mouse NaPiIIb R₅₂₆) has also been reported as being involved in cotransporter function (Hernando et al., J. Membr. Biol., 168:275-282 (1999)).

The interpretation of chemical modification experiments of the Na⁺/phosphate cotransporter has been limited by low cotransporter abundance in the brush border membrane and the large size of the cotransporter. Although brush border membrane vesicles are at least 90% right-side-out, low cotransporter abundance limits structural studies in the native membrane due to the number of competing proteins. Structural studies following cotransporter purification requires detergent removal and membrane reconstitution which introduces variables due to protein orientation and protein degradation. The large size of the intestinal Na⁺/phosphate cotransporter has limited interpretation of chemical modification studies and analysis of ion-induced conformational changes. The intestinal Na⁺/phosphate cotransporter has been identified as a 110-120 kDa polypeptide (Peerce, J. Membr. Biol., 110:189-197 (1989); Hilfiker et al., Proc. Natl. Acad. Sci. (USA), 95:14564-14569 (1998); and Hattenhauer et al., Am. J. Physiol., 277:G756-G762 (1999)) and modeled as containing 8 or 11 transmembrane domains and multiple potential glycosylation sites (Hilfiker et al., Proc. Natl. Acad. Sci. (USA), 95:14564-14569 (1998); Xu et al., Genomics, 62:281-284 (1999); and Field et al., Biochem. Biophys. Res. Comm., 258:578-582 (1999)).

Analysis of the amino acid residues and domains involved in ion binding and transport would be simplified by decreasing the cotransporter mass while retaining function. A similar approach has proven successful with Band 3 (Jennings et al., J. Biol. Chem., 259:4652-4660 (1984); Matsuyama et al., J. Biol. Chem., 258:15376-15381 (1983); and Steck et al., Biochemistry, 17:1216-1222 (1976)), and the Na⁺/K⁺ ATPase (Capasso et al., J. Biol. Chem., 267:1150-1158 (1992); Jorgensen, Acta Physiol. Scand., 146:89-94 (1992); and Shainskaya et al., J. Biol. Chem., 269:10780-10789 (1994)). An additional consideration is that the decreased mass may facilitate structural analysis of the cotransporter, studies of the putative substrate binding sites, and mass spectrometry examination of cotransporter structure.

For all of the above reasons, a need exists for isolated polypeptides having substantial intestinal apical membrane Na⁺/phosphate cotransporter function, and the present invention is directed, in part, to meeting this need.

### SUMMARY OF THE INVENTION

The present invention relates to an isolated NaPiIIb polypeptide.

The present invention also relates to a method for screening a test compound for its ability to bind to a Na⁺/phosphate cotransporter. The method includes providing an isolated NaPiIIb polypeptide; contacting the isolated NaPiIIb polypeptide with the test compound; and determining whether the test compound binds to the isolated NaPiIIb polypeptide.

The present invention also relates to a method for screening a test compound for its ability to affect the function of Na⁺/phosphate cotransporter. The method includes providing an isolated NaPiIIb polypeptide; contacting the isolated NaPiIIb polypeptide with the test compound; and determining whether the test compound affects the function of the isolated NaPiIIb polypeptide.

The present invention also relates to an antibody or fragment thereof specific for a isolated NaPiIIb polypeptide.

### BRIEF DESCRIPTION OF THE DRAWINGS

FIG. 1 is a graph showing HPLC resolution of papain digestion fragments of a Na⁺/phosphate cotransporter.

FIG. 2 is an image of a urea gel electrophoresis (stained with coomassie blue) of HPLC fraction 1.

FIGS. 3A and 3B are images of coomassie blue stainings of fraction 2 (FIG. 3A) and fraction 3 (FIG. 3B) from a Sephadex G-75 column following urea gel electrophoresis.

FIGS. 4A and 4B are graphs showing the effect of various substrates on P40 tryptophan fluorescence emission.

FIG. 5 is a graph showing [³²P] phosphate uptake by full length proteoliposome reconstituted cotransporter and by proteoliposome reconstituted P40 in the presence of Na⁺ and in the presence of K⁺.

FIG. 6 is a qraph showing the effect of pH on P40 activity.

### DETAILED DESCRIPTION OF THE INVENTION

One aspect of the present invention relates to an isolated NaPiIIb polypeptide.

As used herein, a NaPiIIb polypeptide is a polypeptide (i) which has an amino acid sequence corresponding to a portion of the intestinal brush border membrane Na⁺/phosphate cotransporter sequence and (ii) which has substantial Na⁺/phosphate cotransporter function. For the purposes of the present invention, polypeptides which have substantial Na⁺/phosphate cotransporter function are meant to include those peptides which have greater than about 20% (e.g., greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, and/or greater than about 95%) of the Na⁺/phosphate cotransporter activity of intact intestinal brush border membrane Na⁺/phosphate cotransporter, as measured, for example, by reconstituting the isolated polypeptide into prteoliposomes and measuring Na⁺-selective [³²P] phosphate transport, as described (for example) further below.

The phrase “isolated” when referring to a polypeptide, means a chemical composition which is not contained in an organism or an organism's cell in which it is naturally found. The isolated polypeptide can be “purified”, i.e., substantially free from other biological components. Preferably, the polypeptide is in a homogeneous state, which is meant to include homogeneous dry (e.g., lyophilized) polypeptides or homogeneous polypeptides in aqueous solution. Purity and homogeneity are typically determined using analytical chemistry techniques such as polyacrylamide gel electrophoresis or high performance liquid chromatography. A polypeptide which is the predominant species present in a preparation is, for the purposes of the present invention, to be considered substantially purified. Generally, a purified, isolated polypeptide will comprise more than 80% of all macromolecular species present in the preparation. Preferably, the polypeptide is purified such that it represents greater than 90% of all macromolecular species present. More preferably the polypeptide is purified to greater than 95%, and most preferably the polypeptide is purified to substantial homogeneity, wherein other macromolecular species are not detected by conventional techniques. “Purified” and “isolated” polypeptides of the present invention can be synthetically or chemically produced, or they can be isolated from mixtures of materials produced by digestion of naturally occurring materials.

Illustratively, the NaPiIIb polypeptide of the present invention can have a molecular weight of less than 110 kDa, such as less than about 100 kDa, less than about 90 kDa, less than about 80, less than about 70, less than about 60, less than about 50, from about 5 to about 100, from about 10 to about 90, from about 20 to about 80, from about 25 to about 70, from about 30 to about 60, from about 35 to about 55, from about 35 to about 45, and/or about 40 kDa.

The isolated NaPiIIb polypeptide of the present invention can be a polypeptide which comprises an amino acid sequence corresponding to SEQ ID NO: 1 (XAKYRWFAVFYLIFF); it can be a polypeptide which comprises an amino acid sequence of SEQ ID NO: 1; it can be a polypeptide which comprises an amino acid sequence corresponding to SEQ ID NO: 2 (AKYRWFAVFYLIFF); it can be a polypeptide which comprises an amino acid sequence of SEQ ID NO: 2; it can be a polypeptide which comprises an amino acid sequence corresponding to SEQ ID NO: 3 (SAKYRWFAVFYLIFF); it can be a polypeptide which comprises an amino acid sequence of SEQ ID NO: 3; it can be a polypeptide which comprises an amino acid sequence corresponding to SEQ ID NO: 4 (SAKYRWFAVFYLIIF); and/or it can be a polypeptide which comprises an amino acid sequence of SEQ ID NO: 4.

Additionally or alternatively, the isolated NaPiIIb polypeptide of the present invention can be a polypeptide which does not comprise an amino acid sequence of SEQ ID NO: 5 (XVNFVLPDLAVGILL); it can be a polypeptide which does not comprise an amino acid sequence of SEQ ID NO: 6 (VNFVLPDLAVGILL); it can be a polypeptide which does not comprise an amino acid sequence of SEQ ID NO: 7 (VNFSLPDLAVGILL); and/or it can be a polypeptide which does not comprise an amino acid sequence of SEQ ID NO: 8 (VNFHLPDLAVGTILL).

Still additionally or alternatively, the isolated NaPiIIb polypeptide of the present invention can be a polypeptide which does not comprise an amino acid sequence of SEQ ID NO: 9 (PSYXWTDGIQT); it can be a polypeptide which does not comprise an amino acid sequence of SEQ ID NO: 10 (PSYWTDGIQT); it can be a polypeptide which does not comprise an amino acid sequence of SEQ ID NO: 11 (PSYCWTDGIQT); and/or it can be a polypeptide which does not comprise an amino acid sequence of SEQ ID NO: 12 (PSLCWTDGIQN).

For example, the isolated NaPiIIb polypeptide of the present invention can be a polypeptide which comprises an amino acid sequence corresponding to SEQ ID NO: 1 but which does not comprise an amino acid sequence of SEQ ID NO: 5 and which does not comprise an amino acid sequence of SEQ ID NO: 9; it can be a polypeptide which comprises an amino acid sequence corresponding to SEQ ID NO: 1 but which does not comprise an amino acid sequence of SEQ ID NO: 5 and which does not comprise an amino acid sequence of SEQ ID NO: 10; it can be a polypeptide which comprises an amino acid sequence corresponding to SEQ ID NO: 1 but which does not comprise an amino acid sequence of SEQ ID NO: 5 and which does not comprise an amino acid sequence of SEQ ID NO: 11; it can be a polypeptide which comprises an amino acid sequence corresponding to SEQ ID NO: 1 but which does not comprise an amino acid sequence of SEQ ID NO: 5 and which does not comprise an amino acid sequence of SEQ ID NO: 12; it can be a polypeptide which comprises an amino acid sequence corresponding to SEQ ID NO: 1 but which does not comprise an amino acid sequence of SEQ ID NO: 6 and which does not comprise an amino acid sequence of SEQ ID NO: 9; it can be a polypeptide which comprises an amino acid sequence corresponding to SEQ ID NO: 1 but which does not comprise an amino acid sequence of SEQ ID NO: 6 and which does not comprise an amino acid sequence of SEQ ID NO: 10; it can be a polypeptide which comprises an amino acid sequence corresponding to SEQ ID NO: 1 but which does not comprise an amino acid sequence of SEQ ID NO: 6 and which does not comprise an amino acid sequence of SEQ ID NO: 11; it can be a polypeptide which comprises an amino acid sequence corresponding to SEQ ID NO: 1 but which does not comprise an amino acid sequence of SEQ ID NO: 6 and which does not comprise an amino acid sequence of SEQ ID NO: 12; it can be a polypeptide which comprises an amino acid sequence corresponding to SEQ ID NO: 1 but which does not comprise an amino acid sequence of SEQ ID NO: 7 and which does not comprise an amino acid sequence of SEQ ID NO: 9; it can be a polypeptide which comprises an amino acid sequence corresponding to SEQ ID NO: 1 but which does not comprise an amino acid sequence of SEQ ID NO: 7 and which does not comprise an amino acid sequence of SEQ ID NO: 10; it can be a polypeptide which comprises an amino acid sequence corresponding to SEQ ID NO: 1 but which does not comprise an amino acid sequence of SEQ ID NO: 7 and which does not comprise an amino acid sequence of SEQ ID NO: 11; it can be a polypeptide which comprises an amino acid sequence corresponding to SEQ ID NO: 1 but which does not comprise an amino acid sequence of SEQ ID NO: 7 and which does not comprise an amino acid sequence of SEQ ID NO: 12; it can be a polypeptide which comprises an amino acid sequence corresponding to SEQ ID NO: 1 but which does not comprise an amino acid sequence of SEQ ID NO: 8 and which does not comprise an amino acid sequence of SEQ ID NO: 9; it can be a polypeptide which comprises an amino acid sequence corresponding to SEQ ID NO: 1 but which does not comprise an amino acid sequence of SEQ ID NO: 8 and which does not comprise an amino acid sequence of SEQ ID NO: 10; it can be a polypeptide which comprises an amino acid sequence corresponding to SEQ ID NO: 1 but which does not comprise an amino acid sequence of SEQ ID NO: 8 and which does not comprise an amino acid sequence of SEQ ID NO: 11; and/or it can be a polypeptide which comprises an amino acid sequence corresponding to SEQ ID NO: 1 but which does not comprise an amino acid sequence of SEQ ID NO: 8 and which does not comprise an amino acid sequence of SEQ ID NO: 12.

As further example, the isolated NaPiIIb polypeptide of the present invention can be a polypeptide which comprises an amino acid sequence corresponding to SEQ ID NO: 2 but which does not comprise an amino acid sequence of SEQ ID NO: 5 and which does not comprise an amino acid sequence of SEQ ID NO: 9; it can be a polypeptide which comprises an amino acid sequence corresponding to SEQ ID NO: 2 but which does not comprise an amino acid sequence of SEQ ID NO: 5 and which does not comprise an amino acid sequence of SEQ ID NO: 10; it can be a polypeptide which comprises an amino acid sequence corresponding to SEQ ID NO: 2 but which does not comprise an amino acid sequence of SEQ ID NO: 5 and which does not comprise an amino acid sequence of SEQ ID NO: 11; it can be a polypeptide which comprises an amino acid sequence corresponding to SEQ ID NO: 2 but which does not comprise an amino acid sequence of SEQ ID NO: 5 and which does not comprise an amino acid sequence of SEQ ID NO: 12; it can be a polypeptide which comprises an amino acid sequence corresponding to SEQ ID NO: 2 but which does not comprise an amino acid sequence of SEQ ID NO: 6 and which does not comprise an amino acid sequence of SEQ ID NO: 9; it can be a polypeptide which comprises an amino acid sequence corresponding to SEQ ID NO: 2 but which does not comprise an amino acid sequence of SEQ ID NO: 6 and which does not comprise an amino acid sequence of SEQ ID NO: 10; it can be a polypeptide which comprises an amino acid sequence corresponding to SEQ ID NO: 2 but which does not comprise an amino acid sequence of SEQ ID NO: 6 and which does not comprise an amino acid sequence of SEQ ID NO: 11; it can be a polypeptide which comprises an amino acid sequence corresponding to SEQ ID NO: 2 but which does not comprise an amino acid sequence of SEQ ID NO: 6 and which does not comprise an amino acid sequence of SEQ ID NO: 12; it can be a polypeptide which comprises an amino acid sequence corresponding to SEQ ID NO: 2 but which does not comprise an amino acid sequence of SEQ ID NO: 7 and which does not comprise an amino acid sequence of SEQ ID NO: 9; it can be a polypeptide which comprises an amino acid sequence corresponding to SEQ ID NO: 2 but which does not comprise an amino acid sequence of SEQ ID NO: 7 and which does not comprise an amino acid sequence of SEQ ID NO: 10; it can be a polypeptide which comprises an amino acid sequence corresponding to SEQ ID NO: 2 but which does not comprise an amino acid sequence of SEQ ID NO: 7 and which does not comprise an amino acid sequence of SEQ ID NO: 11; it can be a polypeptide which comprises an amino acid sequence corresponding to SEQ ID NO: 2 but which does not comprise an amino acid sequence of SEQ ID NO: 7 and which does not comprise an amino acid sequence of SEQ ID NO: 12; it can be a polypeptide which comprises an amino acid sequence corresponding to SEQ ID NO: 2 but which does not comprise an amino acid sequence of SEQ ID NO: 8 and which does not comprise an amino acid sequence of SEQ ID NO: 9; it can be a polypeptide which comprises an amino acid sequence corresponding to SEQ ID NO: 2 but which does not comprise an amino acid sequence of SEQ ID NO: 8 and which does not comprise an amino acid sequence of SEQ ID NO: 10; it can be a polypeptide which comprises an amino acid sequence corresponding to SEQ ID NO: 2 but which does not comprise an amino acid sequence of SEQ ID NO: 8 and which does not comprise an amino acid sequence of SEQ ID NO: 11; and/or it can be a polypeptide which comprises an amino acid sequence corresponding to SEQ ID NO: 2 but which does not comprise an amino acid sequence of SEQ ID NO: 8 and which does not comprise an amino acid sequence of SEQ ID NO: 12.

As still further example, the isolated NaPiIIb polypeptide of the present invention can be a polypeptide which comprises an amino acid sequence corresponding to SEQ ID NO: 3 but which does not comprise an amino acid sequence of SEQ ID NO: 5 and which does not comprise an amino acid sequence of SEQ ID NO: 9; it can be a polypeptide which comprises an amino acid sequence corresponding to SEQ ID NO: 3 but which does not comprise an amino acid sequence of SEQ ID NO: 5 and which does not comprise an amino acid sequence of SEQ ID NO: 10; it can be a polypeptide which comprises an amino acid sequence corresponding to SEQ ID NO: 3 but which does not comprise an amino acid sequence of SEQ ID NO: 5 and which does not comprise an amino acid sequence of SEQ ID NO: 11; it can be a polypeptide which comprises an amino acid sequence corresponding to SEQ ID NO: 3 but which does not comprise an amino acid sequence of SEQ ID NO: 5 and which does not comprise an amino acid sequence of SEQ ID NO: 12; it can be a polypeptide which comprises an amino acid sequence corresponding to SEQ ID NO: 3 but which does not comprise an amino acid sequence of SEQ ID NO: 6 and which does not comprise an amino acid sequence of SEQ ID NO: 9; it can be a polypeptide which comprises an amino acid sequence corresponding to SEQ ID NO: 3 but which does not comprise an amino acid sequence of SEQ ID NO: 6 and which does not comprise an amino acid sequence of SEQ ID NO: 10; it can be a polypeptide which comprises an amino acid sequence corresponding to SEQ ID NO: 3 but which does not comprise an amino acid sequence of SEQ ID NO: 6 and which does not comprise an amino acid sequence of SEQ ID NO: 11; it can be a polypeptide which comprises an amino acid sequence corresponding to SEQ ID NO: 3 but which does not comprise an amino acid sequence of SEQ ID NO: 6 and which does not comprise an amino acid sequence of SEQ ID NO: 12; it can be a polypeptide which comprises an amino acid sequence corresponding to SEQ ID NO: 3 but which does not comprise an amino acid sequence of SEQ ID NO: 7 and which does not comprise an amino acid sequence of SEQ ID NO: 9; it can be a polypeptide which comprises an amino acid sequence corresponding to SEQ ID NO: 3 but which does not comprise an amino acid sequence of SEQ ID NO: 7 and which does not comprise an amino acid sequence of SEQ ID NO: 10; it can be a polypeptide which comprises an amino acid sequence corresponding to SEQ ID NO: 3 but which does not comprise an amino acid sequence of SEQ ID NO: 7 and which does not comprise an amino acid sequence of SEQ ID NO: 11; it can be a polypeptide which comprises an amino acid sequence corresponding to SEQ ID NO: 3 but which does not comprise an amino acid sequence of SEQ ID NO: 7 and which does not comprise an amino acid sequence of SEQ ID NO: 12; it can be a polypeptide which comprises an amino acid sequence corresponding to SEQ ID NO: 3 but which does not comprise an amino acid sequence of SEQ ID NO: 8 and which does not comprise an amino acid sequence of SEQ ID NO: 9; it can be a polypeptide which comprises an amino acid sequence corresponding to SEQ ID NO: 3 but which does not comprise an amino acid sequence of SEQ ID NO: 8 and which does not comprise an amino acid sequence of SEQ ID NO: 10; it can be a polypeptide which comprises an amino acid sequence corresponding to SEQ ID NO: 3 but which does not comprise an amino acid sequence of SEQ ID NO: 8 and which does not comprise an amino acid sequence of SEQ ID NO: 11; and/or it can be a polypeptide which comprises an amino acid sequence corresponding to SEQ ID NO: 3 but which does not comprise an amino acid sequence of SEQ ID NO: 8 and which does not comprise an amino acid sequence of SEQ ID NO: 12.

As yet further example, the isolated NaPiIIb polypeptide of the present invention can be a polypeptide which comprises an amino acid sequence corresponding to SEQ ID NO: 4 but which does not comprise an amino acid sequence of SEQ ID NO: 5 and which does not comprise an amino acid sequence of SEQ ID NO: 9; it can be a polypeptide which comprises an amino acid sequence corresponding to SEQ ID NO: 4 but which does not comprise an amino acid sequence of SEQ ID NO: 5 and which does not comprise an amino acid sequence of SEQ ID NO: 10; it can be a polypeptide which comprises an amino acid sequence corresponding to SEQ ID NO: 4 but which does not comprise an amino acid sequence of SEQ ID NO: 5 and which does not comprise an amino acid sequence of SEQ ID NO: 11; it can be a polypeptide which comprises an amino acid sequence corresponding to SEQ ID NO: 4 but which does not comprise an amino acid sequence of SEQ ID NO: 5 and which does not comprise an amino acid sequence of SEQ ID NO: 12; it can be a polypeptide which comprises an amino acid sequence corresponding to SEQ ID NO: 4 but which does not comprise an amino acid sequence of SEQ ID NO: 6 and which does not comprise an amino acid sequence of SEQ ID NO: 9; it can be a polypeptide which comprises an amino acid sequence corresponding to SEQ ID NO: 4 but which does not comprise an amino acid sequence of SEQ ID NO: 6 and which does not comprise an amino acid sequence of SEQ ID NO: 10; it can be a polypeptide which comprises an amino acid sequence corresponding to SEQ ID NO: 4 but which does not comprise an amino acid sequence of SEQ ID NO: 6 and which does not comprise an amino acid sequence of SEQ ID NO: 11; it can be a polypeptide which comprises an amino acid sequence corresponding to SEQ ID NO: 4 but which does not comprise an amino acid sequence of SEQ ID NO: 6 and which does not comprise an amino acid sequence of SEQ ID NO: 12; it can be a polypeptide which comprises an amino acid sequence corresponding to SEQ ID NO: 4 but which does not comprise an amino acid sequence of SEQ ID NO: 7 and which does not comprise an amino acid sequence of SEQ ID NO: 9; it can be a polypeptide which comprises an amino acid sequence corresponding to SEQ ID NO: 4 but which does not comprise an amino acid sequence of SEQ ID NO: 7 and which does not comprise an amino acid sequence of SEQ ID NO: 10; it can be a polypeptide which comprises an amino acid sequence corresponding to SEQ ID NO: 4 but which does not comprise an amino acid sequence of SEQ ID NO: 7 and which does not comprise an amino acid sequence of SEQ ID NO: 11; it can be a polypeptide which comprises an amino acid sequence corresponding to SEQ ID NO: 4 but which does not comprise an amino acid sequence of SEQ ID NO: 7 and which does not comprise an amino acid sequence of SEQ ID NO: 12; it can be a polypeptide which comprises an amino acid sequence corresponding to SEQ ID NO: 4 but which does not comprise an amino acid sequence of SEQ ID NO: 8 and which does not comprise an amino acid sequence of SEQ ID NO: 9; it can be a polypeptide which comprises an amino acid sequence corresponding to SEQ ID NO: 4 but which does not comprise an amino acid sequence of SEQ ID NO: 8 and which does not comprise an amino acid sequence of SEQ ID NO: 10; it can be a polypeptide which comprises an amino acid sequence corresponding to SEQ ID NO: 4 but which does not comprise an amino acid sequence of SEQ ID NO: 8 and which does not comprise an amino acid sequence of SEQ ID NO: 11; and/or it can be a polypeptide which comprises an amino acid sequence corresponding to SEQ ID NO: 4 but which does not comprise an amino acid sequence of SEQ ID NO: 8 and which does not comprise an amino acid sequence of SEQ ID NO: 12.

As further illustration, the isolated NaPiIIb polypeptide of the present invention can be a polypeptide which has a molecular weight of less than 110 kDa (e.g., from about 5 kDa to about 100 kDa, from about 35 kDa to about 45 kDa, and/or about 40 kDa) and which (i) comprises an amino acid sequence corresponding to SEQ ID NO: 1, SEQ ID NO: 2, SEQ ID NO: 3, and/or SEQ ID NO: 4; (ii) which does not comprise an amino acid sequence of SEQ ID NO: 5, SEQ ID NO: 6, SEQ ID NO: 7, and/or SEQ ID NO: 8; and/or (iii) which does not comprise an amino acid sequence of SEQ ID NO: 9, SEQ ID NO: 10, SEQ ID NO: 11, and/or SEQ ID NO: 12.

It will be readily understood by those skilled in the art and it is intended here that, when reference is made to particular sequence listings, such reference includes sequences which substantially correspond to those described including allowances for minor sequencing errors, single amino acid changes, deletions, substitutions and the like. Further, it will be understood that the polypeptides of the present invention can contain naturally-occurring or non-naturally-occurring amino acids, including the D-form of the amino acids, amino acid derivatives, and amino acid mimics and mimetics. The choice of including an (L)- or a (D)-amino acid in the polypeptides depends, in part, on the desired characteristics of the polypeptide. The polypeptides of the present invention may also be cyclized. As used herein, the terms “amino acid mimic” and “mimetic” mean an amino acid analog or non-amino acid moiety that has the same or similar functional characteristic of a given amino acid. For instance, an amino acid mimic of a hydrophobic amino acid is one which is non-polar and retains hydrophobicity, generally by way of containing an aliphatic chemical group. By way of further example, an arginine mimic can be an analog of arginine which contains a side chain having a positive charge at physiological pH, as is characteristic of the guanidinium side chain reactive group of arginine. In addition, modifications to the polypeptide backbone and polypeptide bonds thereof are also encompassed within the scope of amino acid mimic or mimetic. Such modifications can be made to the amino acid, derivative thereof, non-amino acid moiety, or the polypeptide either before or after the amino acid, derivative thereof or non-amino acid moiety is incorporated into the polypeptide. What is critical is that such modifications mimic the polypeptide backbone and bonds which make up the same and have substantially the same spatial arrangement and distance as is typical for traditional peptide bonds and backbones. An example of one such modification is the reduction of the carbonyl(s) of the amide peptide backbone to an amine. A number of reagents are available and well known for the reduction of amides to amines such as those disclosed in Wann et al., J. Org. Chem., 46:257 (1981) and Raucher et al., Tetrahedron Lett., 21:14061 (1980). An amino acid mimic is, therefore, an organic molecule that retains the similar amino acid pharmacophore groups as is present in the corresponding amino acid and which exhibits substantially the same spatial arrangement between functional groups. The substitution of amino acids by non-naturally occurring amino acids and amino acid mimics as described above can enhance the overall activity or properties of an individual polypeptide based on the modifications to the backbone or side chain functionalities. For example, these types of alterations to the amino acid substituents and polypeptides can enhance the polypeptide's stability to enzymatic breakdown. Modifications to the polypeptide backbone similarly can add stability and enhance activity.

More particularly, as used herein, “a polypeptide which comprises an amino acid sequence of” a specified sequence is meant to include only those, polypeptides which include the exact specified sequence. As used herein, “a polypeptide comprising an amino acid sequence corresponding to” a specified sequence is meant to include those polypeptides which include the exact specified sequence as well as those polypeptides which include sequences having substantial identity with the specified sequence and those polypeptides which include sequences having substantial homology with the specified sequence.

The following terms are used to describe the sequence relationships between two or more amino acid sequences of polypeptides: “reference sequence”, “comparison window”, “sequence identity”, “sequence homology”, “percentage of sequence identity”, “percentage of sequence homology”, “substantial identity”, and “substantial homology”. A “reference sequence” is a defined sequence used as a basis for a sequence comparison; a reference sequence may be a subset of a larger sequence.

Optimal alignment of sequences for aligning a comparison window may be conducted, for example, by the local homology algorithm of Smith et al., Adv. Appl. Math., 2:482-489 (1981) and Smith et al., J. Mol. Biol., 147:195-197 (1981); by the homology alignment algorithm of Needleman et al., J. Mol. Biol., 48:443-453 (1970); by the search for similarity method of Pearson et al., Proc. Natl. Acad. Sci. USA, 85:2444-2448 (1988); or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.).

As applied to polypeptides, the terms “substantial identity” or “substantial sequence identity” mean that two peptide sequences, when optimally aligned, such as by the programs GAP or BESTFIT using default gap, share at least 90 percent sequence identity, preferably at least 95 percent sequence identity, more preferably at least 96, 97, 98 or 99 percent sequence identity.

“Percentage amino acid identity” or “percentage amino acid sequence identity” refers to a comparison of the amino acids of two polypeptides which, when optimally aligned, have approximately the designated percentage of the same amino acids. For example, “95% amino acid identity” refers to a comparison of the amino acids of two polypeptides which when optimally aligned have 95% amino acid identity. Preferably, residue positions which are not identical differ by conservative amino acid substitutions. For example, the substitution of amino acids having similar chemical properties such as charge or polarity are not likely to affect the properties of a protein. Examples include glutamine for asparagine or glutamic acid for aspartic acid.

As further applied to polypeptides, the terms “substantial homology” or “substantial sequence homology” mean that two peptide sequences, when optimally aligned, such as by the programs GAP or BESTFIT using default gap, share at least 90 percent sequence homology, preferably at least 95 percent sequence homology, more preferably at least 96, 97, 98 or 99 percent sequence homology.

“Percentage amino acid homology” or “percentage amino acid sequence homology” refers to a comparison of the amino acids of two polypeptides which, when optimally aligned, have approximately the designated percentage of the same amino acids or conservatively substituted amino acids. For example, “95% amino acid homology” refers to a comparison of the amino acids of two polypeptides which when optimally aligned have 95% amino acid homology. As used herein, homology refers to identical amino acids or residue positions which are not identical but differ only by conservative amino acid substitutions. For example, the substitution of amino acids having similar chemical properties such as charge or polarity are not likely to affect the properties of a protein. Examples include glutamine for asparagine or glutamic acid for aspartic acid.

One skilled in the art, using the above sequences or formulae, can readily synthesize the polypeptides of the present invention. Standard procedures for preparing synthetic polypeptides are well known in the art. For example, the novel polypeptides can be synthesized using: the solid phase peptide synthesis (SPPS) method of Merrifield (J. Am. Chem. Soc., 85:2149-2154 (1964)) or modifications of SPPS; or the peptides can be synthesized using standard solution methods well known in the art (see, for example, Bodanzsky, Principles of Peptide Synthesis, 2nd revised ed., Berlin-New York: Springer-Verlag (1988 and 1993)). Alternatively, simultaneous multiple peptide synthesis (SMPS) techniques well known in the art can be used. Peptides prepared by the method of Merrifield can be synthesized using an automated peptide synthesizer such as the Applied Biosystems 431A-01 Peptide Synthesizer (Mountain View, Calif.) or using the manual peptide synthesis technique described in Houghten, Proc. Natl. Acad. Sci., USA, 82:5131-5135 (1985).

Alternatively, the polypeptides of the present invention can be produced by other methods, such as by isolation from mixtures of materials produced by digestion of naturally occurring materials (e.g., produced by digestion of intestinal brush border membrane Na⁺/phosphate cotransporter). Illustratively, the polypeptides of the present invention can be produced from intestinal brush border membrane Na⁺/phosphate cotransporter by papain digestion. Suitable Na⁺/phosphate cotransporter:papain weight ratios can range from about 20:1 to about 80:1, such as from about 30:1 to about 70:1, from about 40:1 to about 60:1, from about 45:1 to about 55:1, and/or about 50:1. Suitable digestion times can range from about 20 minutes to about 120 minutes, such as from about 30 minutes to about 90 minutes and/or about 60 minutes. The polypeptides of the present invention can then be isolated from the resulting mixture of digestion products by conventional procedures, such as those described in the examples set forth hereinbelow.

The polypeptides of the present invention can be used to screen test compounds for their ability to bind to or otherwise affect the function of intestinal brush border membrane Na⁺/phosphate cotransporter. The method includes providing an isolated NaPiIIb polypeptide; contacting the isolated NaPiIIb polypeptide with the test compound; and determining whether the test compound binds to or otherwise affects the function of isolated NaPiIIb polypeptide. Since the isolated NaPiIIb polypeptides of the present invention retain the function of intact intestinal brush border membrane Na⁺/phosphate cotransporter, the effects of the test compound on isolated NaPiIIb polypeptide can be correlated to or otherwise used to determine whether and/or to what extent the test compound binds to or otherwise affects the function of intestinal brush border membrane Na⁺/phosphate cotransporter.

Whether the function of isolated NaPiIIb polypeptide is affected by a test compound can be determined directly in accordance with conventional procedures for a measuring Na⁺-selective [³²P] phosphate transport. Suitable procedures are described in the examples set forth hereinbelow and in International Publication No. WO 00/43402. Alternatively, whether the function of isolated NaPiIIb polypeptide is affected by a test compound (and, hence, whether the function of intestinal brush border membrane Na⁺/phosphate cotransporter would be affected by the test compound) can be inferred from studies which assess whether the test compound binds to the isolated NaPiIIb polypeptide.

Compounds identified as having the ability to bind to or otherwise affect the function of intestinal brush border membrane Na⁺/phosphate cotransporter in accordance with the method of the present invention can be used to inhibit sodium-mediated phosphate uptake, to reduce serum PTH, calcium, calcitriol, or phosphate, and/or to treat renal disease in patients, as described, for example, in International Publication No. WO 00/43402.

The isolated NaPiIIb polypeptides of the present invention can also be used to further identify and/or characterize the binding site of intestinal brush border membrane Na⁺/phosphate cotransporter, for example by probing an isolated NaPiIIb polypeptide with an inhibitor of intestinal brush border membrane Na⁺/phosphate cotransporter, such as 2′-phosphophloretin or other inhibitors of intestinal apical membrane Na⁺/phosphate cotransportation described in International Publication No. WO 00/43402.

The present invention further relates to an antibody or fragment thereof specific for an isolated NaPiIIb polypeptide of the present invention. Antibodies of the subject invention include polyclonal antibodies and monoclonal antibodies capable of binding to the polypeptides of the present invention, as well as fragments of these antibodies, and humanized forms. Humanized forms of the antibodies of the subject invention may be generated using one of the procedures known in the art such as chimerization. Fragments of the antibodies of the present invention include, but are not limited to, the Fab, the F(ab′)₂, and the Fc fragments. Suitable antibodies or fragments thereof include those which are specific for an isolated NapiIIb polypeptide of the present invention but which do not bind to intestinal brush border membrane Na⁺/phosphate cotransporter as well as those which are specific for an isolated NaPiIIb polypeptide of the present invention and which also bind to intestinal brush border membrane Na⁺/phosphate cotransporter.

The invention also provides hybridomas which are capable of producing the above-described antibodies. A hybridoma is an immortalized cell line which is capable of secreting a specific monoclonal antibody.

In general, techniques for preparing polyclonal and monoclonal antibodies as well as hybridomas capable of producing the desired antibody are well known in the art (e.g., see Campbell, Monoclonal Antibody Technology: Laboratory Techniques in Biochemistry and Molecular Biology, Amsterdam, The Netherlands: Elsevier Science Publishers (1984) and St. Groth et al., J. Immunol. Methods, 35:1-21 (1980) (“Campbell”)). Any animal (mouse, rabbit, etc.) which is known to produce antibodies can be immunized with the antigenic polypeptides of the present invention (or an antigenic fragment thereof). Methods for immunization are well known in the art. Such methods include subcutaneous or intraperitoneal injection of the polypeptide. One skilled in the art will recognize that the amount of the polypeptide used for immunization will vary based on the animal which is immunized, the antigenicity of the polypeptide, and the site of injection.

The polypeptide which is used as an immunogen may be modified or administered in an adjuvant in order to increase the polypeptide's antigenicity. Methods of increasing the antigenicity of a polypeptide are well known in the art and include, but are not limited to, coupling the antigen with a heterologous protein (such as a globulin or beta-galactosidase) or through the inclusion of an adjuvant during immunization.

For monoclonal antibodies, spleen cells from the immunized animals are removed, fused with myeloma cells, such as SP2/O-Ag 15 myeloma cells, and allowed to become monoclonal antibody producing hybridoma cells.

Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell. Res., 175:109-124 (1988)).

Hybridomas secreting the desired antibodies are cloned and the class and subclass are determined using procedures known in the art (see, e.g., Campbell).

For polyclonal antibodies, antibody containing antisera is isolated from the immunized animal and is screened for the presence of antibodies with the desired specificity using one of the above-described procedures.

In accordance with the above discussion, the subject invention further provides a method of producing an antibody specific for a polypeptide of the present invention in a host. The method comprises selecting the isolated polypeptide of the present invention or an antigenic portion thereof and introducing the selected polypeptide of the present invention or antigenic portion thereof into a host to induce production of an antibody specific for polypeptide of the present invention in the host.

The present invention also relates to the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.), fluorescent labels (such as FITC or rhodamine, etc.), paramagnetic atoms, etc. Procedures for accomplishing such labeling are well known in the art (for example, see Sternberger et al., J. Histochem. Cytochem., 18:315-333 (1970); Bayer et al., Meth. Enzym., 62:308-315 (1979); Engvall et al., J. Immunol., 109:129-135 (1972); and Goding, J. Immunol. Meth., 13:215-226 (1976)).

The labeled antibodies or fragments thereof of the present invention can be used for in vitro, in vivo, and in situ assays to identify cells or tissues which express intestinal brush border membrane Na⁺/phosphate cotransporter, to identify samples containing polypeptides of the present invention, or to detect the presence of polypeptides of the present invention in a sample. More particularly, the antibodies or fragments thereof can thus be used to detect the presence of polypeptides of the present invention in a sample, by contacting the sample with the antibody or fragment thereof. The antibody or fragment thereof binds to polypeptides of the present invention present in the sample, forming a complex therewith. The complex can then be detected, thereby detecting the presence of polypeptides of the present invention in the sample. As will be readily apparent to those skilled in the art, such a method could also be used quantitatively to assess the amount of polypeptide of the present invention in a sample. The antibodies or fragments thereof of the present invention can also be used to study the intestinal brush border membrane Na⁺/phosphate cotransporter's binding site (or the binding site of an isolated NaPiIIb polypeptide), for example, by interfering (e.g., sterically) with the intestinal brush border membrane Na⁺/phosphate cotransporter's binding (or the isolated NaPiIIb polypeptide's binding) of 2′-phosphophloretin or other inhibitors of intestinal apical membrane Na⁺/phosphate cotransportation.

The present invention, in yet another aspect thereof, relates to polypeptides that bind specifically to an antibody that binds specifically to an isolated NaPiIIb polypeptide of the present invention. These polypeptides can be used, for example, as a positive control in an assay which utilizes the antibody. Illustratively, the subject polypeptide can be a non-naturally-occurring polypeptide, and/or it can be one which binds specifically to an antibody that binds specifically to an isolated NaPiIIb polypeptide which (i) has a molecular weight of about 40 kDa; (ii) comprises an amino acid sequence corresponding to SEQ ID NO: 1, SEQ ID NO: 2, SEQ ID NO: 3, and/or SEQ ID NO: 4; (iii) does not comprise an amino acid sequence of SEQ ID NO: 5, SEQ ID NO: 6, SEQ ID NO: 7, and/or SEQ ID NO: 8; and (iv) does not comprise an amino acid sequence of SEQ ID NO: 9, SEQ ID NO: 10, SEQ ID NO: 11, and/or SEQ ID NO: 12.

The present invention is further illustrated with the following examples.

### EXAMPLES

### Example 1—Overview

Polypeptide fragments of the rabbit intestinal brush border membrane Na⁺/phosphate cotransporter were generated by limited proteolytic digestion of the purified cotransporter. Polypeptide fragments of the intestinal Na⁺/phosphate cotransporter were compared to the intact Na⁺/phosphate cotransporter for substrate-induced conformational changes, and Na⁺ and H₂PO₄⁻ occlusion. Following liposome reconstitution, Na⁺-dependent phosphate uptake was also determined. Substrate-induced conformational changes and ion occlusion were similar in the intact Na⁺/phosphate cotransporter and a 40 kDa papain digestion fragment of the Na⁺/phosphate cotransporter. Na⁺-dependent phosphate uptake by the liposome reconstituted 40 kDa papain digestion fragment was similar to the intact liposome reconstituted cotransporter, but reduced in transport rate. These results are consistent with the 40 kDa papain digestion fragment being a transport competent Na⁺ and phosphate transporter similar to the 110-120 kDa intestinal Na⁺/phosphate cotransporter.

### Example 2—Materials and Methods

SDS-PAGE supplies were purchased from Biorad, Hercules, Calif. [³²P] phosphate and [²²Na] were purchased from DuPont/NEN, Boston, Mass. CHAPS, n-octyl glucoside, DTT, urea, Hepes, Tris, and MES were purchased from Sigma Chemical Co., St Louis, Mo. Polybuffer 74, Sephadex and Sephacryl were purchased from Amersham Pharmacia Biotech, Piscataway, N.J. Dowex cation and anion exchange resins were purchased from Aldrich Chemical Co., Milwaukee, Wis. All other chemicals were purchased from Fisher Scientific, Houston, Tex. and were reagent grade or better. CHAPS and n-octyl glucoside were recrystallized once from ethanol. All other chemicals were used as received from the suppliers.

Ca²⁺-precipitated brush border membranes were prepared as follows. Rabbit intestinal brush border membrane vesicles (BBMV) were prepared by divalent metal precipitation and differential centrifugation as previously described (Stevens et al., J. Membr. Biol., 66:213-225 (1982); and Peerce et al., Am. J. Physiol., 264:G609-G616 (1993)). Following purification, the vesicles were re-suspended in 300 mM mannitol and 10 mM Hepes/Tris pH 7.5, and stored in liquid N₂ until needed. Purification of BBMV protein was assayed using the brush border membrane markers sucrase (Dahlquist, Anal. Biochem., 7:18-25 (1964)), and alkaline phosphatase (Hanna et al., J. Supramolec. Struct., 11:451-466 (1979)). During the course of these studies the enrichment of the BBMV protein varied between 24-fold and 32-fold relative to the initial intestinal homogenate.

BBMV protein was further purified by centrifugation through 5 ml. disposable syringes packed with Sephadex G-25 (Peerce et al., Am. J. Physiol., 264:G609-G616 (1993); and Penefsky, J. Biol. Chem., 252:2891-2899 (1977)). Briefly Sephadex G-25 was equilibrated with 150 mM KCl, 2 mM EDTA, 10 mM Hepes/Tris pH 7.5, and 1 mM DTT. Prior to addition to the columns, Ca²⁺-BBMV protein was incubated in the same media for 15 minutes at 8° C. at 10 mg/ml. Immediately prior to addition of protein, the columns were centrifuged for 15 minutes at 2500 g to remove water. Protein was layered on the Sephadex, and the centrifugation repeated. Protein was collected, diluted 40 times in 300 mM mannitol and 10 mM Hepes/Tris pH 7.5, and centrifuged at 48,000 g for 40 minutes. This step was repeated twice to ensure removal of the DTT which, can interfere with protein assays. The final pellets were re-suspended in 300 mM mannitol and 10 mM Hepes/Tris pH 7.5, and they were stored in liquid N₂ until needed.

Intestinal Na⁺/phosphate cotransporter was purified as follows. The intestinal Na⁺/phosphate cotransporter was purified from Sephadex treated BBMV protein as previously described (Peerce, Biochim. Biophys. Acta, 1323:45-56 (1997); and Peerce et al., Am. J. Physiol., 264:G609-G616 (1993)). Cotransporter enrichment was assayed by SDS-PAGE according to the method of Laemmli (Laemmli, Nature (London), 227:680-685 (1971)) and by [²²Na] occlusion. [²²Na] occlusion was performed as previously described (Peerce, Biochim. Biophys. Acta, 1323:45-56 (1997); and Peerce, Kid. Int., 49:988-991 (1996)), and the enrichment was measured as the increase in [²²Na] occluded per milligram protein compared to partially purified Na⁺/phosphate cotransporter (initial chromatofocusing chromatography column fraction eluting between pH 4.8 and pH 4.4). A 4 to 6 fold increase in [²²Na] occluded per milligram of protein compared to the partially purified cotransporter yielded a protein fraction, which was a single band on analytical SDS-PAGE gels. Assuming 2 moles of Na⁺ bind per mole of cotransporter protein, 86%±6% (n=4) of the recovered cotransporter retained activity.

Proteolytic enzyme digestion of the Na⁺/phosphate cotransporter was carried out as follows. Papain digestion of the purified cotransporter was performed in 50 mM Tris-Cl pH 7, 0.1 mM DTT, 0.5% CHAPS, and 1 mM EDTA (Digestion buffer) as previously described (Jennings et al., J. Biol. Chem., 259:4652-4660 (1984); and Peerce, Biochim. Biophys. Acta, 1239:1-10 (1995)). Prior to the addition of protein, papain was activated by pre-incubation in the digestion buffer for 30 minutes at 4° C. The protein to papain ratio was either 50:1 or 20:1. Papain digestion was carried out at 37° C. for 1 hour to 4 hours. The digestion was stopped by addition of a 10-fold excess of iodoacetate. For electrophoretic analysis an aliquot of the mixture was precipitated with 90% acetone at 4° C. The acetone-precipitated protein was washed with water and centrifuged at 2500 g for 30 minutes. This step was repeated twice. Alternatively, the mixture was dialyzed against 150 mM KCl and 10 mM Hepes/Tris pH 7.5 for 24 hours at 4° C. The mixture was then frozen in 150 mM KCl, 10 mM Hepes/Tris pH 7.5, and 10% glycerol and stored at liquid N₂ temperatures until needed.

Chymotrypsin and trypsin digestions of the Na⁺/phosphate cotransporter were performed 100 mM NH₄HCO₃ pH 8.3 and 0.1 mM CaCl₂. Digestion was performed at a protein:protease ratio of 50:1 to 10:1. Reaction time was varied between 1 hr and 4 hrs. The reaction was stopped with a 10-fold excess of soybean trypsin inhibitor and processed as described above. Na⁺/phosphate cotransporter was also digested with S. aureus V-8 protease in 100 mM NH₄HCO₃ pH 8 or 100 mM potassium phosphate pH 7.8. Digestion was performed at a 20:1 protein:protease ratio for 2 hours or 12 hours. Digestion was stopped by addition of a 10-fold excess of diisopropyl fluorophosphate. Proteolytic enzyme digestion of the cotransporter was examined by urea gel electrophoresis using the Tris-phosphate buffer system (Kawano et al., J. Biochem., 100:191-199 (1986)).

Cotransporter digestion was analyzed by coomassie blue staining, eosin absorbance, or fluorescein absorption on a Gilford Spectrophotometer. Fluorescein absorbance was analyzed at 483 nm, and eosin absorbance was monitored at 525 nm. Coomassie blue staining was analyzed by scanning densitometry at 595 nm.

Peptides generated for amino acid sequencing were isolated as water-soluble peptides following papain digestion. Na⁺/Phosphate cotransporter was digested with papain for one hour at 37° C. at a protein:papain ratio of 20:1. The resultant peptides were diluted 20-fold with 10 mM Tris-Cl pH 7 and washed through an Amicon flow cell with a 50 kDa cut-off filter. The eluate was lyophilized and dialyzed through dialysis tubing with a 1 kDa cut-off against 10 mM Tris-Cl pH 7. Dialysis was continued for 48 hours with 4 buffer changes. The dialyzed peptides were lyophilized and dialysis and lyophilization repeated. The peptides were resolved by HPLC on a Waters Bondapak C₁₈ column using a 0.1% TFA: 0.1% TFA/25% acetonitrile linear gradient, diluted with water, and lyophilized. Peptides were resuspended in 0.1% TFA and centrifuged at 148,000 g for 1 hour. The supernatants were injected into the HPLC and eluted from the column at a flow rate of 1 ml/min. Peptide fractions were monitored at 215 nm or 280 nm. Peptide fractions were collected and lyophilized. Fractions were resuspended in 25% methanol and dotted onto PVDF paper.

Internal peptides from papain digestion of P40 and P24 were generated by in situ gel fragment digestion with chymotrypsin. Following papain digestion, the polypeptide fragments were resolved by preparative urea gel electrophoresis (Penefsky, J. Biol. Chem., 252:2891-2899 (1977)). The gel was stained with coomassie blue. The selected fragment was cut from the gel and washed with 200 mM NH₄HCO₃/50% CH₃CN, 200 mM NH₄HCO₃/50% CH₃CN+1 mM DTT, 200 mM NH₄HCO₃/50% CH₃CN+1 mM DTT+1 mM iodoacetic acid, and dried. Dried gel slices were hydrated with 200 mM NH₄HCO₃ and incubated with 30 μg/ml chymotrypsin in 200 mM NH₄HCO₃ for 24 hours at 37° C. Peptides were extracted with 0.1% TFA/60% CH₃CN and lyophilized. Lyophilized peptides were resuspended in 0.05% TFA/25% CH₃CN and resolved by HPLC on the Waters C₁₈ column. HPLC elution was monitored at 215 nm. Peptide peaks were lyophilized, resuspended in 25% methanol and dotted unto PVDF squares for sequencing. Amino acid sequencing of Na⁺/phosphate cotransporter peptides was performed on an Applied Biosystem 475A Automatic Amino Acid Sequencer.

Gel filtration of papain digestion fragments was carried out using the following procedure. For experiments requiring purified papain digestion fragments, the fragments were resolved by gel filtration on a 0.75 cm×25 cm Sephadex G-75 column equilibrated with 0.1% n-octyl glucoside 0.25 M KCl, and 40 mM Tris-Cl pH 7 (gel filtration buffer). 2-4 mg of protein was applied to the column in the gel filtration buffer and the column run at 5 ml/hr. Column fractions were assayed at 280 nm and by urea gel electrophoresis (Kawano et al., J. Biochem., 100:191-199 (1986)). Samples for urea gel electrophoresis were prepared by acetone precipitation of an aliquot of the column fraction as previously described (Landolt-Marticorena et al., Molec. Membr. Biol., 12:173-182 (1995)) with one modification. Following the initial centrifugation of the acetone denatured peptides, the peptides were re-suspended three times in water and pelleted by centrifugation at 2500 g to remove detergent.

Na⁺/phosphate cotransporter tryptophan fluorescence and papain fragment tryptophan fluorescence studies were performed on an SLM SPF 500 c spectrofluorimeter (Peerce, Am. J. Physiol., 256:G645-G652 (1989); Peerce, J. Membr. Biol., 110:189-197 (1989); and Peerce, Biochim. Biophys. Acta, 1323:45-56 (1997)). Tryptophan fluorescence was excited at 290 nm and emission was recorded at 350 nm or emission was recorded as a function of emission wavelength between 300 nm and 450 nm. The response of tryptophan fluorescence to substrates was examined in 500 mM KCl, 25 mM Tris-Cl pH 7, and 0.1% n-octyl glucoside. The effect of Na⁺ and phosphate on tryptophan fluorescence was examined in 0.5 M KCl, 0.1 M NaCl, 25 mM Tris-Cl pH 7, and 0.1% n-octyl glucoside.

In some experiments, the effect of Na⁺ on the fluorescence of fluorescein isothiocyanato-phenylglyoxal (FITC-PG) was examined. The Na⁺/phosphate cotransporter was labeled with FITC-PG in 50 mM potassium borate buffer pH 7.4 and 0.1% n-octyl glucoside as previously described (Peerce, Am. J. Physiol., 256:G645-G652 (1989); Peerce, J. Membr. Biol., 110:189-197 (1989); and Peerce, Biochim. Biophys. Acta, 1323:45-56 (1997)). Soluble protein was separated from free FITC-PG by centrifugation through a centricon filter with a 100 kDa cut-off filter. Centrifugation was performed at 2500 g for 30 minutes. The filter was washed 3 times with 25 mM Tris-Cl pH 7, and finally re-suspended in 0.5 M KCl, 25 mM Tris-Cl pH 7, and 0.1% n-octylglucoside.

In some experiments, the effect of pH on Na⁺-induced tryptophan fluorescence quench was examined. In these experiments, protein was diluted into 500 mM KCl, 0.1% n-octyl glucoside, and 25 mM Mes/Tris pH 5.5 to pH 6.5, 25 mM Pipes/Tris pH 7 and pH 7.5, 25 mM Hepes/Tris pH 8, or 25 mM TAPS/Cl pH 8.5 and pH 9. Tryptophan fluorescence was excited at 290 nm, and tryptophan fluorescence emission was recorded as a function of wavelength from 300 nm to 450 nm. 50 mM NaCl was added from a 2 M stock, and tryptophan fluorescence recorded. The results are reported as the change in fluorescence divided by the initial fluorescence and corrected for dilution.

In some experiments, the effect of sulfate on tryptophan fluorescence was examined. In these experiments protein was diluted into 500 mM KCl, 100 mM NaCl, 25 mM Pipes/Tris pH 7, and 0.1% n-octyl glucoside. 1 mM potassium sulfate or 1 mM potassium phosphate was added, and tryptophan fluorescence as a function of wavelength as described above. In some experiments, the effect of phosphate and sulfate on tryptophan fluorescence was recorded as a function of time, and the change in fluorescence/the initial fluorescence (ΔF/Fₒ) was recorded following correction for dilution artifacts.

[²²Na⁺] occlusion by the Na⁺/phosphate cotransporter and its papain-generated fragments was performed using the Light-activated Microsecond (LAM) Timer as previously described (Peerce, Biochim. Biophys. Acta, 1323:45-56 (1997); and Peerce, Kid. Int., 49:988-991 (1996)). Briefly, 1 cc disposable syringes were filled with the Dowex-50W cation exchange resin which had been equilibrated with 10 mM NaCl or 10 mM KCl, 40 mM Tris-Cl pH 7, and 0.1% n-octyl glucoside. Protein or peptides were equilibrated with 100 μM [²²Na], 40 mM Tris-Cl pH 7, and 0.1% n-octyl glucoside by incubation at 4° C. for 10 minutes. Isotope-equilibrated protein was applied to the top of the Dowex column, and a vacuum applied. Protein-retained counts were determined by liquid scintillation counting, protein eluting from the column was determined by SDS-micro Lowry protein assay (Peterson, Anal. Biochem., 100:201-220 (1979)), and column residence time was read from the LAM digital read-out. The amount of occluded Na⁺ was determined from the difference between protein retained counts in Na⁺-equilibrated columns (corrected for protein lost) and counts eluting from the column in the absence of protein (zero protein control).

[³²P] Phosphate occlusion by the Na⁺/phosphate cotransporter and papain-generated peptides was performed as described above for Na⁺ occlusion. Protein was equilibrated with 10 mM NaCl, 10 μM [³²P] phosphate, 40 mM Tris-Cl pH 7, and 0.1% n-octyl glucoside for 10 minutes at 4° C. Equilibrated protein was applied to the anion exchange resin Dowex-1X in 1 cc disposable syringes equilibrated with 10 mM NaCl, 1 mM phosphate, 40 mM Tris-Cl pH 7, and 0.1% n-octyl glucoside. Protein-retained counts were defined as above corrected for protein loss and zero protein controls.

High-pressure liquid chromatography (HPLC) of the Na⁺/phosphate cotransporter and its proteolytic enzyme digestion fragments was performed on a Waters HPLC and a 0.5 cm×30 cm TSK 300 column. The column was equilibrated with 50 mM Tris-Cl pH 7, 0.3 M KCl, and 0.1% n-octyl glucoside. Protein was applied at a final concentration of 1-2 mg/ml, and an isocratic gradient of 50 mM Tris-Cl pH 7, 0.1% n-octyl glucoside, and 0.3 M KCl was used to elute the protein at a flow rate of 0.5 ml/min. Protein was monitored at 280 nm. In some experiments, fluorescein fluorescence was simultaneously monitored using a Spectroflow fluorescence detector with a 480 nm band pass filter at the emission end.

Proteoliposome reconstitution of the papain-generated Na⁺/phosphate cotransporter fragments was carried out by the following procedure. Gel filtration purified fractions containing papain digestion fragments were reconstituted into phosphatidyl choline:cholesterol liposomes as previously described (Peerce et al., Am. J. Physiol., 264:G609-G616 (1993)). Gel filtration column fraction 2, containing the 40 kDa polypeptide, and fraction 3, containing the 24 kDa polypeptide, were proteoliposome reconstituted. The reconstituted peptides were assayed for Na⁺-dependent [³²P] phosphate uptake using 0.22 μm filters as previously described (Peerce et al., Am. J. Physiol., 264:G609-G616 (1993)). Na⁺-dependent uptake was defined as filter-retained counts in the presence of 100 mM NaCl minus filter-retained counts in the presence of 100 mM KCl. Reconstituted protein was assayed by the SDS micro-Lowry protein assay following addition of 1% SDS, and centrifugation at 146,000 g for 40 minutes (Peterson, Anal. Biochem., 100:201-220 (1979)).

### Example 3—Proteolytic Digestion Studies of the Intestinal Na⁺/Phosphate Cotransporter

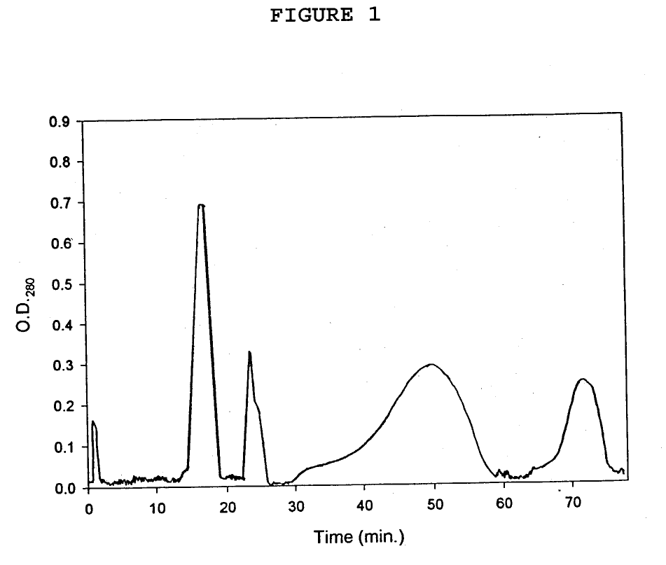

In preliminary experiments, a variety of proteolytic enzymes and digestion reaction conditions were examined. Na⁺/phosphate cotransporter labeled with either FITC-PG (putative phosphate site label (Peerce, Am. J. Physiol., 256:G645-G652 (1989); and Peerce, J. Membr. Biol., 110:189-197 (1989))), or FNAI (putative Na⁺ site label Peerce, J. Membr. Biol., 110:189-197 (1989))) was digested with papain, trypsin, chymotrypsin, or V-8 protease. The reaction conditions varied included reaction time, cotransporter:protease ratio, detergent, and the presence of substrates during enzymatic digestion. Reaction conditions and proteolytic enzymes generating cotransporter polypeptide fractions containing both the phosphate and Na⁺ site labels were selected for further study. Urea gel electrophoresis was used to analyze proteolytic enzyme digestion of the intestinal Na⁺/phosphate cotransporter. Chymotrypsin and trypsin digestion of the Na⁺/phosphate cotransporter in CHAPS or n-octyl glucoside yielded poor digestion at short digestion times (0.5 hr to 2 hr), or no fragment labeled with both ENAI and FITC-PG. V-8 protease digestion yielded small peptide fragments, which could not be resolved by HPLC or SDS-PAGE. Papain digestion at a 50:1 protein to papain ratio yielded 4 polypeptide bands. These bands corresponded to a broad band centered at 40 kDa, a 24 kDa polypeptide band, and 2 broad bands centered at 20 kDa and 14 kDa (results not shown). The papain digestion of the Na⁺/phosphate cotransporter was partially resolved by HPLC. HPLC resolution of papain digestion fragments of the Na⁺/phosphate cotransporter is shown in FIG. 1.

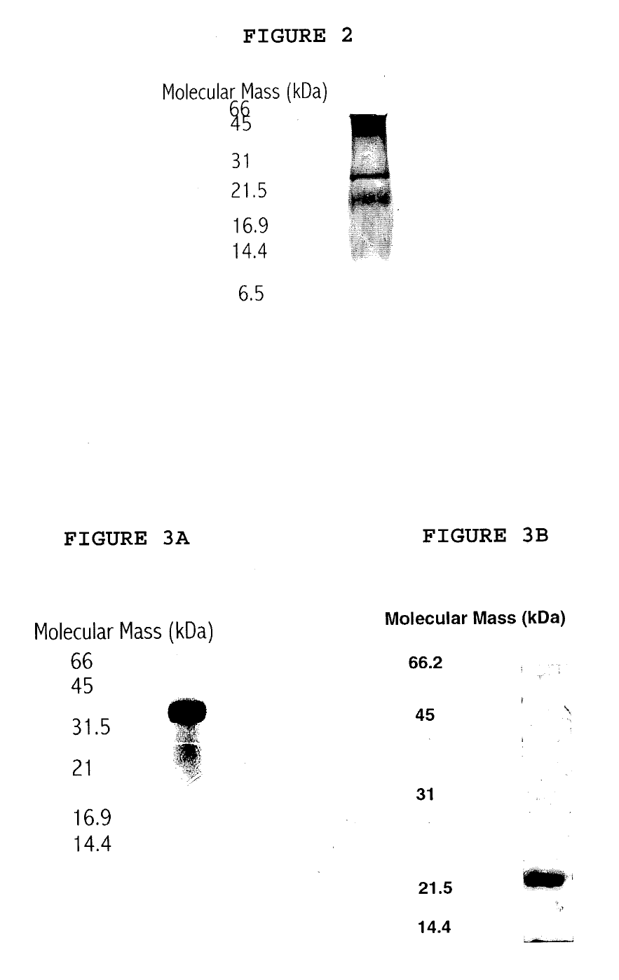

Papain digested Na⁺/phosphate cotransporter was resolved into 4 fractions by HPLC. Based on preliminary experiments, peak 1 contained the FITC-PG and FNAI binding sites. No other fraction contained both FITC-PG and FNAI labeled peptides. Further analysis of peak 1 was performed by urea gel electrophoresis. Urea gel electrophoresis of HPLC fraction 1 is shown in FIG. 2.

FIG. 2 is the coomassie blue stain of HPLC fraction 1. FIG. 2 indicates that HPLC fraction 1 contained 2 major polypeptides. These 2 peptides have apparent molecular masses of 40 kDa and 24 kDa. The relative abundance of the 40 kDa papain polypeptide (P40) to the 24 kDa papain polypeptide (P24) was dependent on the protein to papain ratio used during proteolytic digestion and digestion time. Papain digestion of the purified Na⁺/phosphate cotransporter at a 20:1 ratio of cotransporter to papain altered the ratio of P40 to P24. Papain digestion performed at a ratio of 20:1 resulted in a 75%±15% (n=3) decrease in the 40 kDa peptide and a corresponding increase in the 24 kDa peptide (results not shown). Longer digestion times also increased the percentage of the 24 kDa polypeptide. Papain digestion for 120 minutes increased the percentage of P24 from 22%±8% (n=3) to 58%±10 (n=3). Decreasing the digestion time did not increase the percentage of P40 but did increase the percentage of undigested cotransporter. Papain digestion of the Na⁺/phosphate cotransporter at a protein:papain ratio of 20:1 yielded a number of small water-soluble peptides. These peptides were partially resolved by HPLC on the C₁₈ column. Multiple HPLC runs were required to purify the peptides, resulting in low peptide recoveries. Two peptides were sequenced and the results are shown in Table 1.

TABLE 1
Amino Acid Sequence of Water-Soluble Papain Fragments
from the Rabbit Na⁺/Phosphate Cotransporter

| | | |
|---|---|---|
| Water-soluble | _VNFVLPDLAVGILL | (SEQ ID NO: 5) |
| fragment 5: | | |
| Mouse¹ | ₃₅₆VNFSLPDLAVGILL₃₆₉ | (SEQ ID NO: 7) |
| Human² | ₃₅₄VNFHLPDLAVGTILL₃₆₈ | (SEQ ID NO: 8) |
| Water-soluble | PSY_WTDGIQT | (SEQ ID NO: 9) |
| fragment 6: | | |
| Mouse¹ | ₃₂₅PSYCWTDGIQT₃₃₆ | (SEQ ID NO: 11) |
| Human² | ₃₂₄PSLCWTDGIQN₃₃₅ | (SEQ ID NO: 12) |

¹Hilfiker et al., Proc. Natl. Acad. Sci. (USA), 95:14564-14569 (1998)
²Xu et al., Genomics, 62:281-284 (1999)

In Table 1, the blank space in water-soluble fragment 6 indicates that the amino acid could not be determined, and the blank space in water-soluble fragment 5 indicates that the first amino acid could not be assigned. Table 1 shows the consensus sequences of water-soluble fragment 5 and water-soluble fragment 6. For comparison the deduced amino acid sequences or human and mouse NaPiIIb are shown.

The 40 kDa (P40) and 24 kDa (P24) polypeptides were partially resolved by low pressure chromatography on a Sephadex G-75 column developed with 0.1% N-octyl glucoside, 0.250 M KCl, and 40 mM Tris-Cl pH 7. FIG. 3A is the coomassie blue staining of fraction 2 from the Sephadex G-75 column following urea gel electrophoresis. Scanning densitometry at 595 nm indicated that P40 was 85%±5% (n=5) of the protein found in fraction 2 from the Sephadex G-75 column.

Fraction 3 from the Sephadex G-75 column was also analyzed by SDS-PAGE gel electrophoresis and scanning densitometry. FIG. 3B is the coomassie blue stain of fraction 3 following SDS-PAGE. Scanning densitometry indicated that Sephadex G-75 fraction 3 was 80%+9% (n=5) P24.

### Example 4—Na⁺-Induced Conformational Changes in Papain Fragments of the Na⁺/Phosphate Cotransporter

Na⁺-induced conformational changes, measured by Na⁺-induced FITC-PG fluorescence quenching or Na⁺-induced tryptophan fluorescence quenching in the Na⁺/phosphate cotransporter and the papain generated polypeptides, are summarized in Table 2.

TABLE 2
Effect of Papain Digestion on
Na⁺/Phosphate Cotransporter Activity

| | Na⁺-Dependent | | [³²P] |
|---|---|---|---|
| | FPG | [²²Na] | Phosphate |
| | Fluorescence | Occluded | Occluded |
| Fraction | Quench (%) | (nmoles/mg) | (nmoles/mg) |
| Na⁺/Phosphate | 32 ± 3 | 16 ± 3 | 8 ± 1.2 |
| Cotransporter | | | |
| 40 kDa Papain | 26 ± 3 | 22 ± 4 | 12 ± 2 |
| Polypeptide | | | |
| 24 kDa Papain | Not measurable | 18 ± 6 | 0.4 ± 0.2 |
| Polypeptide | | | |

The results presented in Table 2 are means ±S.E. of 8 determinations of Na⁺-induced FPG fluorescence quenching, 4 determinations of Na occlusion, and 3 determinations of phosphate occlusion. The Na⁺ concentration dependence of Na⁺-induced FITC-PG fluorescence quenching of P40 was fitted to the Hill equation. The apparent Kₘ for Na⁺ was 25 mM±5 mM (n=5). The Hill coefficient, n_{H}, was 2.2±0.3. Similar experiments with the Na⁺/phosphate cotransporter were fitted to an apparent Kₘ of 22 mM±2 mM, and a n_{H} of 1.8±0.2 (n=3). P24 did not have measurable FITC-PG fluorescence.

Table 2 also summarizes experiments examining Na⁺ and phosphate occlusion by papain digestion fragments. Compared to the intact cotransporter, papain digestion resulted in a significant reduction in ion occlusion. [²²Na⁺] retained by P40 was 50% less than the predicted amount of Na⁺ bound based on 2 moles of Na⁺ occluded per mole of Na⁺/phosphate cotransporter. [²²Na] occluded per mole P24 was 22% of the predicted value assuming 2 Na⁺'s per P24.

The 40 kDa papain polypeptide retained Na⁺-dependent [³²P] phosphate occlusion. The rate of loss [³²P] phosphate was faster than in the intact cotransporter (results not shown). Na⁺-dependent [³²P] phosphate occlusion could not be measured in P24.

### Example 5—Substrate-Induced Conformational Changes in the Papain Polypeptide Fragments Measured by Tryptophan Fluorescence

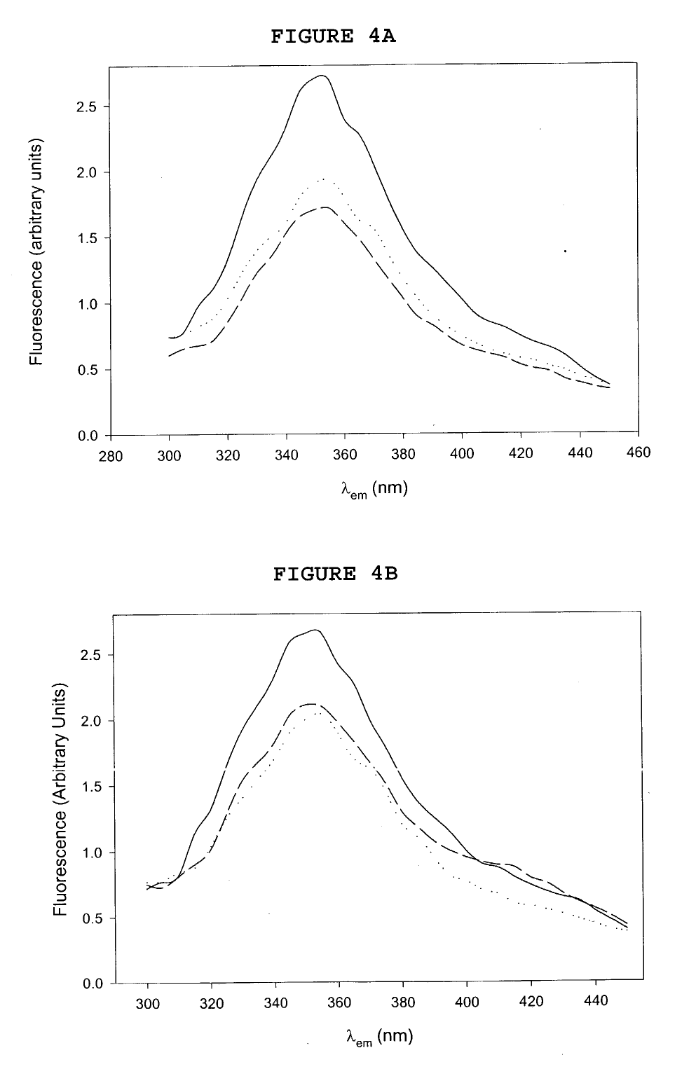

The effect of substrates on P40 tryptophan fluorescence emission is shown in FIGS. 4A and 4B. FIG. 4A shows the tryptophan fluorescence emission as a function of emission wavelength in the presence of 0.5 M KCl (solid line), following the addition of 100 mM NaCl (dotted line), and following the addition of 1 mM phosphate (dashed line). Addition of Na⁺ resulted in a 28%±6% (n=25) tryptophan fluorescence quench and a slight red shift (5 nm to 7 nm). Addition of 1 mM potassium phosphate resulted in an additional 12%±3% (n=8) tryptophan fluorescence quenching. FIG. 4B shows a parallel experiment substituting 10 mM sulfate for phosphate. In the experiment shown, addition of Na⁺ resulted in a 24% fluorescence quenching (dotted line). Following the addition of Na⁺, addition of 10 mM potassium sulfate (dashed line) did not alter tryptophan fluorescence. These results are consistent with P40 retaining Na⁺ and phosphate selectivity. Studies of the effect of substrates on tryptophan fluorescence of the papain digestion fragments are summarized in Table 3.

TABLE 3
Effect of Substrates on Tryptophan Fluorescence
of Papain Digestion Fragments

| | | | number of |
|---|---|---|---|
| Fragment | Addition | ΔF/Fₒ | experiments |
| 40 kDa | Na⁺ | 28 ± 6 | 25 |
| 24 kDa | Na⁺ | 21 ± 3 | 12 |
| 40 kDa | Na⁺ + potassium | 15 ± 2 | 10 |
| | difluorophosphate | | |
| 24 kDa | Na⁺ + potassium | 0.5 ± 0.3 | 8 |
| | difluorophosphate | | |

### Example 6—Proteoliposome Reconstitution of Papain Digestion Fragments

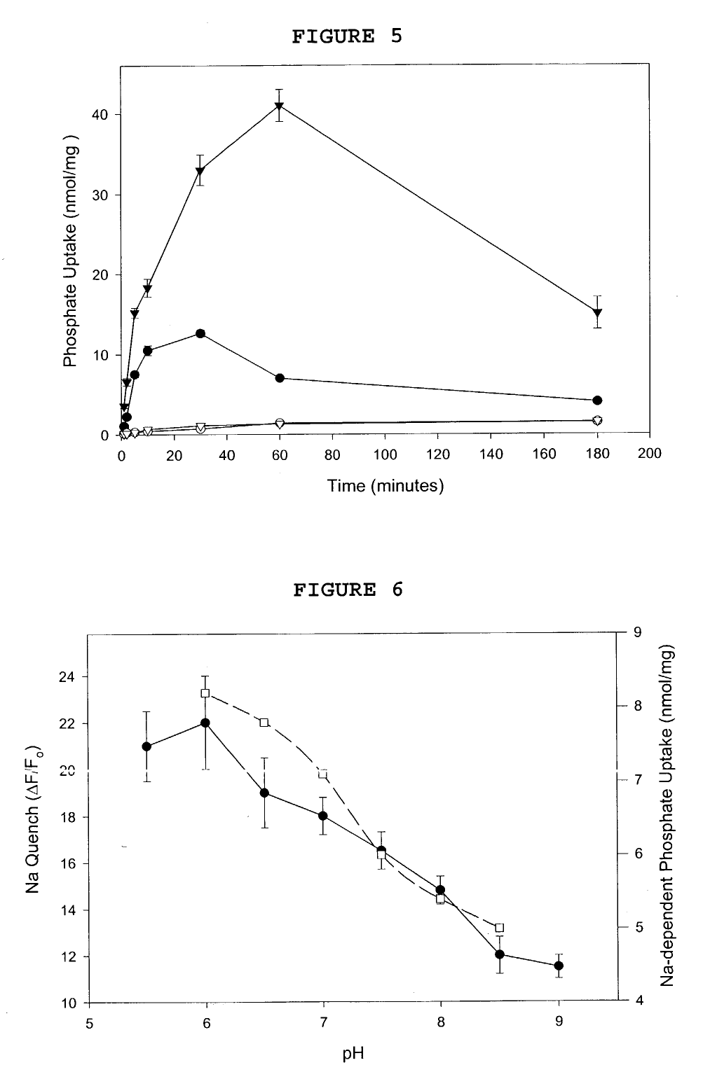

Intact Na⁺/phosphate cotransporter and P40 were each proteoliposome reconstituted as described in Example 2. The results of a typical experiment is shown in FIG. 5.

FIG. 5 shows [³²P] phosphate uptake by full length proteoliposome reconstituted cotransporter (triangles) and proteoliposome reconstituted P40 (circles) in the presence of Na⁺ (closed circles and closed triangles) and in the presence of K⁺ (open triangles and open circles). Liposome reconstituted full-length cotransporter (solid triangles) and liposome reconstituted 40 kDa papain fragment displayed overshoot phenomena. Na⁺-dependent phosphate uptake by proteoliposome reconstituted intact cotransporter was 20-fold (24±4, n=3) over equilibrium phosphate uptake. Na⁺-dependent phosphate uptake by reconstituted P40 was 8-fold (8±2, n=3) larger than equilibrium phosphate uptake. Na⁺-independent phosphate uptakes (open symbols) were similar for the 40 kDa papain digestion fragment (open circles) and the full-length cotransporter (open triangles). P24 did not reconstitute into cholesterol:phosphatidyl choline liposomes (results not shown).

The effect of pH on P40 activity is shown in FIG. 6. Referring to FIG. 6, the Na⁺-induced conformational change as assayed by the Na⁺-induced tryptophan fluorescence quenching in P40 (solid circles, solid line) decreased with increasing pH. The apparent pK_{A} was 7.4±0.2 (n=3). Tryptophan fluorescence emission of P40 at 350 nm in 500 mM KCl, 0.05% n-octyl glucoside, 10 μg of P40, and 40 mM buffer was determined as described in Example 2. 50 mM NaCl was added and tryptophan fluorescence emission at 350 nm was determined. In a parallel experiment the change in tryptophan fluorescence due to dilution was determined by adding an equal volume of KCl. The effect of NaCl on tryptophan fluorescence emission was normalized for the initial fluorescence. Referring still to FIG. 6, Na⁺-dependent phosphate uptake into proteoliposome reconstituted P40 (open squares, dashed line) also decreased with increasing pH. The apparent pK_{A} was 7.6±0.4 (n=3). Na⁺-dependent uptake was defined as uptake in the presence of 50 mM NaCl, or 50 mM KCl, 100 mM mannitol, 40 mM buffer, and 50 μM [³²P] phosphate. Uptakes were performed for 2 minutes at 23° C. Results are means ±S.E. of triplicate determinations and representative of 3 experiments.

### Example 7—Amino Acid Sequence of P40

Amino acid sequencing studies of P40 and P24 did not yield complete sequences. The peptides appeared to be N-terminal blocked. In order to overcome this problem, P40 was digested in situ in the gel following purification by polyacrylamide gel electrophoresis in 8M urea (Kawano et al., J. Biochem., 100:191-199 (1986)). Internal sequence from P40 is shown in Table 4.

TABLE 4

Internal Amino Acid Sequence from P40

| | | |
|---|---|---|
| P40 | _AKYRWFAVFYLIFF | (SEO ID NO: 1) |
| Mouse¹ | ₅₂₂SAKYRWFAVFYLIFF₅₃₆ | (SEQ ID NO: 3) |
| Human² | ₅₂₁SAKYRWFAVFYLIIF₅₃₅ | (SEQ ID NO: 4) |

¹Hilfiker et al., Proc. Natl. Acad. Sci. (USA), 95:14564-14569 (1998)
²Xu et al., Genomics, 62:281-284 (1999)

Table 4 shows an internal sequence from P40 following extensive chymotryptic digestion in situ in the gel. For comparison the human and mouse amino acid sequences are also shown.

### Example 8—Discussion of Results

The intestinal brush border membrane Na⁺/phosphate cotransporter was digested with papain, and the resulting digestion fragments were examined for cotransporter-related activity. Five assays were used to examine activities associated with cotransporter function for comparison to the intact cotransporter. These included: Na⁺-induced conformational changes, (Na⁺+potassium difluorophosphate, or Na⁺+phosphate)-induced conformational changes, Na⁺ occlusion, Na⁺-dependent phosphate occlusion, and Na⁺-dependent phosphate uptake following proteoliposome reconstitution. Substrate specificity and pH sensitivity of P40 were also examined. The results suggest that a 40 kDa polypeptide generated by papain digestion was capable of Na⁺ and phosphate cotransport similar to the intact intestinal brush border membrane cotransporter.

Papain digestion of the detergent-solubilized Na⁺/phosphate cotransporter yielded multiple (at least 7 polypeptides) polypeptides varying in apparent molecular mass from 40 kDa to less than 4 kDa. HPLC partially resolved these peptides. A single HPLC fraction (fraction 1) retained substrate site labels. HPLC fraction 1 contained 2 peptides, a 40 kDa polypeptide and a 24 kDa polypeptide.

Two results suggest that the 24 kDa papain fragment resulted from digestion of the 40 kDa papain fragment. The relative ratio of the 40 kDa polypeptide to the 24 kDa polypeptide decreased as a function of papain digestion time and as a function of papain concentration. At a 50:1 cotransporter to papain ratio, the 24 kDa polypeptide was 25%±5% (n=3) of the 40 kDa polypeptide. Papain digestion at a 20:1 cotransporter to papain ratio resulted in an increase in the amount of 24 kDa polypeptide and a corresponding decrease in the amount of the 40 kDa polypeptide. At a 20:1 cotransporter to papain ratio, the 24 kDa polypeptide was 85%±6% (n=3) of the 40 kDa polypeptide. The second result suggesting that the 24 kDa polypeptide was derived from the 40 kDa polypeptide was the observation that P40 and P24 contained similar amounts of the Na⁺ site label, FNAI. Both polypeptides also retained Na⁺-selective conformational changes, and retained partial Na⁺ occlusion.

In contrast to the results with Na⁺, only the 40 kDa polypeptide responded to phosphate in a Na⁺ selective manner. The 40 kDa polypeptide retained tryptophan fluorescence quenching upon addition of potassium difluorophosphate to detergent-solubilized polypeptide in the presence of Na⁺. The 40 kDa polypeptide retained FITC-PG (fluorescein isothiocayanto-phenylglyoxal) binding. In addition, only the 40 kDa polypeptide occluded phosphate in the presence of Na⁺. Na⁺induced and (Na⁺+phosphate)-induced tryptophan fluorescence quenching were similar in the intact Na⁺/phosphate cotransporter and in P40 (Table 3). In contrast ion occlusion was reduced following papain digestion. The intact Na⁺/phosphate cotransporter retained between 80% of the predicted Na⁺ occlusion for a 110 kDa to 120 kDa polypeptide with 2 Na⁺'s bound per cotransporter. Na⁺ occlusion by P40 was 40% of the predicted amount of Na⁺ bound by a 40 kDa polypeptide with 2 Na⁺'s bound per P40. Phosphate bound to P40 showed a similar reduction (44%±4%) in the number of moles of phosphate bound per mole P40. This reduction in activity could be the result of decreased stability of the Na⁺ conformation, increased P40 denaturation, or increased P40 aggregation.

The observation that the 40 kDa papain-generated polypeptide reconstituted into proteoliposomes transported [³²P] phosphate in a Na⁺-selective manner, similar to the intact cotransporter, strongly suggests that the 40 kDa papain-generated polypeptide retained Na⁺ and phosphate sites, and ion selectivity. The full-length cotransporter, and P40 reconstituted into proteolipomes in a similar manner. Per mole polypeptide, twenty percent of the detergent-solubilized Na⁺/phosphate cotransporter (21%±4%, n=3) and P40 (22%+3%, n=3) reconstituted into proteoliposomes. Calculated turnover rates were also similar. The intact cotransporter turned over 0.05 s⁻¹ and P40 turned over 0.03 s⁻¹.

The pH dependence of the Na⁺-induced tryptophan fluorescence quench and Na⁺-dependent phosphate uptake (FIG. 6) was similar to that previously reported for Na⁺-dependent phosphate uptake into intestinal brush border membrane vesicles (Danisi et al., Am. J. Physiol.,246:G180-G186 (1984)) and for phosphate uptake by NaPiIIb expressed in frog oocyte (De la Horra et al., J. Biol. Chem., 275:6284-6287 (2000)). Na⁺-dependent phosphate uptake and the Na⁺-induced conformational change had nearly identical apparent pK_{A}'s. In the absence of a series of experiments examining the effect of pH on the (Na⁺+phosphate)-induced tryptophan fluorescence quench, Na⁺ off rate, phosphate off rate, and phosphate on rate, it is not possible to assign a significance to the apparent pK_{A}'s calculated from FIG. 6.

Amino acid sequencing of P40 and P24 from their amino termini proved problematic. Both papain peptides appeared to be N-terminal blocked. Urea de-ionization (Marshall et al., pp. 1-66 in Darbre, ed., Practical Protein Chemistry—A Handbook, New York: John Wiley and Sons (1986)) or polyacrylamide gel pretreatment with thioglycolic acid (Moos et al., J. Biol. Chem., 263: 6005-6008 (1988)) did not prevent N-terminal block. Sephadex G-75 purified peptides did not yield sequence N-terminal sequence information. Based on these findings it appears that the peptides were blocked prior to purification.

Internal peptide amino acid sequencing in situ yielded sequence data for P40. Following urea gel purification, chymotryptic cleavage yielded a complex of peptides, which were sequenced following HPLC purification. The internal amino acid sequence of P40 is shown in Table 4. The amino acid sequence shown in Table 4 is consistent with the amino acid sequences of the water-soluble peptides shown in Table 1 and the deduced amino acid sequence of NaPiIIb (Hilfiker et al., Proc. Natl. Acad. Sci. (USA), 95:14564-14569 (1998); and Xu et al., Genomics, 62:281-284 (1999)). The papain peptide purification studies suggest that preparative urea gel purification and in situ secondary digestions (e.g., those including trypsin, V-8 protease, and/or CNBr) can provide further amino acid sequence information.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined, for example, in the claims which follow.

What is claimed is:

1. An isolated NaPiIIb polypeptide.

2. An isolated NaPiIIb polypeptide according to claim 1, wherein said isolated NaPiIIb polypeptide comprises an amino acid sequence corresponding to SEQ ID NO: 1, SEQ ID NO: 2, SEQ ID NO: 3, and/or SEQ ID NO: 4.

3. An isolated NaPiIIb polypeptide according to claim 2, wherein said isolated NaPiIIb polypeptide does not comprise an amino acid sequence of SEQ ID NO: 5, SEQ ID NO: 6, SEQ ID NO: 7, and/or SEQ ID NO: 8.

4. An isolated NaPiIIb polypeptide according to claim 2, wherein said isolated NaPiIIb polypeptide does not comprise an amino acid sequence of SEQ ID NO: 9, SEQ ID NO: 10, SEQ ID NO: 11, and/or SEQ ID NO: 12.

5. An isolated NaPiIIb polypeptide according to claim 2, wherein said isolated NaPiIIb polypeptide does not comprise an amino acid sequence of SEQ ID NO: 5, SEQ ID NO: 6, SEQ ID NO: 7, and/or SEQ ID NO: 8 and wherein said isolated NaPiIIb polypeptide does not comprise an amino acid sequence of SEQ ID NO: 9, SEQ ID NO: 10, SEQ ID NO: 11, and/or SEQ ID NO: 12.

6. An isolated NaPiIIb polypeptide according to claim 1, wherein said isolated NaPiIIb polypeptide comprises an amino acid sequence of SEQ ID NO: 1, SEQ ID NO: 2, SEQ ID NO: 3, and/or SEQ ID NO: 4.

7. An isolated NaPiIIb polypeptide according to claim 1, wherein said isolated NaPiIIb polypeptide does not comprise an amino acid sequence of SEQ ID NO: 5, SEQ ID NO: 6, SEQ ID NO: 7, and/or SEQ ID NO: 8.

8. An isolated NaPiIIb polypeptide according to claim 1, wherein said isolated NaPiIIb polypeptide does not comprise an amino acid sequence of SEQ ID NO: 9, SEQ ID NO: 10, SEQ ID NO: 11, and/or SEQ ID NO: 12.

9. An isolated NaPiIIb polypeptide according to claim 1, wherein said isolated NaPiIIb polypeptide does not comprise an amino acid sequence of SEQ ID NO: 5, SEQ ID NO: 6, SEQ ID NO: 7, and/or SEQ ID NO: 8 and wherein said isolated NaPiIIb polypeptide does not comprise an amino acid sequence of SEQ ID NO: 9, SEQ ID NO: 10, SEQ ID NO: 11, and/or SEQ ID NO: 12.

10. An isolated NaPiIIb polypeptide according to claim 1, wherein said isolated NaPiIIb polypeptide has a molecular weight of about 40 kDa.

11. An isolated NaPiIIb polypeptide according to claim 10, wherein said isolated NaPiIIb polypeptide comprises an amino acid sequence corresponding to SEQ ID NO: 1, SEQ ID NO: 2, SEQ ID NO: 3, and/or SEQ ID NO: 4.

12. An isolated NaPiIIb polypeptide according to claim 11, wherein said isolated NaPiIIb polypeptide does not comprise an amino acid sequence of SEQ ID NO: 5, SEQ ID NO: 6, SEQ ID NO: 7, and/or SEQ ID NO: 8.

13. An isolated NaPiIIb polypeptide according to claim 11, wherein said isolated NaPiIIb polypeptide does not comprise an amino acid sequence of SEQ ID NO: 9, SEQ ID NO: 10, SEQ ID NO: 11, and/or SEQ ID NO: 12.

14. An isolated NaPiIIb polypeptide according to claim 11, wherein said isolated NaPiIIb polypeptide does not comprise an amino acid sequence of SEQ ID NO: 5, SEQ ID NO: 6, SEQ ID NO: 7, and/or SEQ ID NO: 8 and wherein said isolated NaPiIIb polypeptide does not comprise an amino acid sequence of SEQ ID NO: 9, SEQ ID NO: 10, SEQ ID NO: 11, and/or SEQ ID NO: 12.

15. An isolated NaPiIIb polypeptide according to claim 10, wherein said isolated NaPiIIb polypeptide comprises an amino acid sequence of SEQ ID NO: 1, SEQ ID NO: 2, SEQ ID NO: 3, and/or SEQ ID NO: 4.

16. An isolated NaPiIIb polypeptide according to claim 10, wherein said isolated NaPiIIb polypeptide does not comprise an amino acid sequence of SEQ ID NO: 5, SEQ ID NO: 6, SEQ ID NO: 7, and/or SEQ ID NO: 8.

17. An isolated NaPiIIb polypeptide according to claim 10, wherein said isolated NaPiIIb polypeptide does not comprise an amino acid sequence of SEQ ID NO: 9, SEQ ID NO: 10, SEQ ID NO: 11, and/or SEQ ID NO: 12.

18. An isolated NaPiIIb polypeptide according to claim 10, wherein said isolated NaPiIIb polypeptide does not comprise an amino acid sequence of SEQ ID NO: 5, SEQ ID NO: 6, SEQ ID NO: 7, and/or SEQ ID NO: 8 and wherein said isolated NaPiIIb polypeptide does not comprise an amino acid sequence of SEQ ID NO: 9, SEQ ID NO: 10, SEQ ID NO: 11, and/or SEQ ID NO: 12.

19. An isolated NaPiIIb polypeptide according to claim 1, wherein said isolated NaPiIIb polypeptide is prepared by digesting Na⁺/phosphate cotransporter with papain.

20. An isolated NaPiIIb polypeptide according to claim 19, wherein said isolated NaPiIIb polypeptide is prepared by digesting Na⁺/phosphate cotransporter with papain in a Na⁺/phosphate cotransporter:papain weight ratio of greater than 20:1.

21. An isolated NaPiIIb polypeptide according to claim 19, wherein said isolated NaPiIIb polypeptide is prepared by digesting Na⁺/phosphate cotransporter with papain in a Na⁺/phosphate cotransporter:papain weight ratio of about 50:1.

22. A method for screening a test compound for its ability to bind to Na⁺/phosphate cotransporter, said method comprising:
providing an isolated NaPiIIb polypeptide according to claim 1;
contacting the isolated NaPiIIb polypeptide with the test compound; and
determining whether the test compound binds to the isolated NaPiIIb polypeptide.

23. A method according to claim 22, wherein the isolated NaPiIIb polypeptide comprises an amino acid sequence corresponding to SEQ ID NO: 1, SEQ ID NO: 2, SEQ ID NO: 3, and/or SEQ ID NO: 4.

24. A method according to claim 22, wherein the isolated NaPiIIb polypeptide comprises an amino acid sequence of SEQ ID NO: 1, SEQ ID NO: 2, SEQ ID NO: 3, and/or SEQ ID NO: 4.

25. A method according to claim 22, wherein the isolated NaPiIIb polypeptide does not comprise an amino acid sequence of SEQ ID NO: 5, SEQ ID NO: 6, SEQ ID NO: 7, and/or SEQ ID NO: 8.

26. A method according to claim 22, wherein the isolated NaPiIIb polypeptide does not comprise an amino acid sequence of SEQ ID NO: 9, SEQ ID NO: 10, SEQ ID NO: 11, and/or SEQ ID NO: 12.

27. A method according to claim 22, wherein the isolated NaPiIIb polypeptide does not comprise an amino acid sequence of SEQ ID NO: 5, SEQ ID NO: 6, SEQ ID NO: 7, and/or SEQ ID NO: 8 and wherein the isolated NaPiIIb polypeptide does not comprise an amino acid sequence of SEQ ID NO: 9, SEQ ID NO: 10, SEQ ID NO: 11, and/or SEQ ID NO: 12.

28. A method according to claim 22, wherein the isolated NaPiIIb polypeptide has a molecular weight of about 40 kDa.

29. A method according to claim 22, wherein the isolated NaPiIIb polypeptide has a molecular weight of about 40 kDa; wherein the isolated NaPiIIb polypeptide comprises an amino acid sequence corresponding to SEQ ID NO: 1, SEQ ID NO: 2, SEQ ID NO: 3, and/or SEQ ID NO: 4; wherein the isolated NaPiIIb polypeptide does not comprise an amino acid sequence of SEQ ID NO: 5, SEQ ID NO: 6, SEQ ID NO: 7, and/or SEQ ID NO: 8; and wherein the isolated NaPiIIb polypeptide does not comprise an amino acid sequence of SEQ ID NO: 9, SEQ ID NO: 10, SEQ ID NO: 11, and/or SEQ ID NO: 12.

30. A method for screening a test compound for its ability to affect the function of Na⁺/phosphate cotransporter, said method comprising:
providing an isolated NaPiIIb polypeptide according to claim 1;
contacting the isolated NaPiIIb polypeptide with the test compound; and
determining whether the test compound affects the function of the isolated NaPiIIb polypeptide.

31. A method according to claim 30, wherein the isolated NaPiIIb polypeptide comprises an amino acid sequence corresponding to SEQ ID NO: 1, SEQ ID NO: 2, SEQ ID NO: 3, and/or SEQ ID NO: 4.

32. A method according to claim 30, wherein the isolated NaPiIIb polypeptide comprises an amino acid sequence of SEQ ID NO: 1, SEQ ID NO: 2, SEQ ID NO: 3, and/or SEQ ID NO: 4.

33. A method according to claim 30, wherein the isolated NaPiIIb polypeptide does not comprise an amino acid sequence of SEQ ID NO: 5, SEQ ID NO: 6, SEQ ID NO: 7, and/or SEQ ID NO: 8.

34. A method according to claim 30, wherein the isolated NaPiIIb polypeptide does not comprise an amino acid sequence of SEQ ID NO: 9, SEQ ID NO: 10, SEQ ID NO: 11, and/or SEQ ID NO: 12.

35. A method according to claim 30, wherein the isolated NaPiIIb polypeptide does not comprise an amino acid sequence of SEQ ID NO: 5, SEQ ID NO: 6, SEQ ID NO: 7, and/or SEQ ID NO: 8 and wherein the isolated NaPiIIb polypeptide does not comprise an amino acid sequence of SEQ ID NO: 9, SEQ ID NO: 10, SEQ ID NO: 11, and/or SEQ ID NO: 12.

36. A method according to claim 30, wherein the isolated NaPiIIb polypeptide has a molecular weight of about 40 kDa.

37. A method according to claim 30, wherein the isolated NaPiIIb polypeptide has a molecular weight of about 40 kDa; wherein the isolated NaPiIIb polypeptide comprises an amino acid sequence corresponding to SEQ ID NO: 1, SEQ ID NO: 2, SEQ ID NO: 3, and/or SEQ ID NO: 4; wherein the isolated NaPiIIb polypeptide does not comprise an amino acid sequence of SEQ ID NO: 5, SEQ ID NO: 6, SEQ ID NO: 7, and/or SEQ ID NO: 8; and wherein the isolated NaPiIIb polypeptide does not comprise an amino acid sequence of SEQ ID NO: 9, SEQ ID NO: 10, SEQ ID NO: 11, and/or SEQ ID NO: 12.

38. An antibody or fragment thereof specific for a isolated NaPiIIb polypeptide according to claim 1.

39. An antibody or fragment thereof according to claim 38, wherein said antibody or fragment thereof is a Fab fragment, a F(ab′)₂ fragment, or a Fc fragment.

40. An antibody or fragment thereof according to claim 38, wherein said antibody is a polyclonal antibody.

41. An antibody or fragment thereof according to claim 38, wherein said antibody is a monoclonal antibody.

42. A hybridoma which produces monoclonal antibodies according to claim 41.

43. A purified polypeptide that binds specifically to an antibody that binds specifically to antibody or fragment thereof according to claim 38.

44. A purified polypeptide according to claim 43, wherein said polypeptide is a non-naturally-occurring polypeptide.

45. An antibody or fragment thereof specific for a isolated NaPiIIb polypeptide according to claim 14.

46. A purified polypeptide that binds specifically to an antibody that binds specifically to antibody or fragment thereof according to claim 45.

47. A purified polypeptide according to claim 46, wherein said polypeptide is a non-naturally-occurring polypeptide.


SEQUENCE LISTING (12 sequences)

SEQ ID NO: 1 - protein, 15 aa, Rabbit
  MISC_FEATURE (1)..(1): Xaa can be any amino acid
XAKYRWFAVFYLIFF

SEQ ID NO: 2 - protein, 14 aa, Rabbit
AKYRWFAVFYLIFF

SEQ ID NO: 3 - protein, 15 aa, Rabbit
SAKYRWFAVFYLIFF

SEQ ID NO: 4 - protein, 15 aa, Rabbit
SAKYRWFAVFYLIIF

SEQ ID NO: 5 - protein, 15 aa, Rabbit
  MISC_FEATURE (1)..(1): Xaa can be any amino acid
XVNFVLPDLAVGILL

SEQ ID NO: 6 - protein, 14 aa, Rabbit
VNFVLPDLAVGILL

SEQ ID NO: 7 - protein, 14 aa, Rabbit
VNFSLPDLAVGILL

SEQ ID NO: 8 - protein, 15 aa, Rabbit
VNFHLPDLAVGTILL

SEQ ID NO: 9 - protein, 11 aa, Rabbit
  MISC_FEATURE (4)..(4): Xaa can be any amino acid
PSYXWTDGIQT

SEQ ID NO: 10 - protein, 10 aa, Rabbit
PSYWTDGIQT

SEQ ID NO: 11 - protein, 11 aa, Rabbit
PSYCWTDGIQT

SEQ ID NO: 12 - protein, 11 aa, Rabbit
PSLCWTDGIQN